(19) United States
(12) Patent Application Publication
(10) Pub. No.: US 2007/0270971 A1
(43) Pub. Date: Nov. 22, 2007

(54) Intervertebral prosthetic disc with improved wear resistance

(75) Inventors: Hai H. Trieu, Cordova, TN (US); Greg Marik, Memphis, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/375,382

(22) Filed: Mar. 14, 2006

(51) Int. Cl. A61F 2/44

(52) U.S. Cl. 623/17.15

(57) ABSTRACT

An intervertebral prosthetic disc is disclosed and can be installed within an intervertebral space between a superior vertebra and an inferior vertebra. The intervertebral prosthetic disc can include an inferior component having a depression formed therein and a superior component having a projection extending therefrom. The projection can be configured to movably engage the depression and allow relative motion between the inferior component and the superior component. Further, the projection can include a superior wear resistant layer configured to engage the depression.

### FIELD OF THE DISCLOSURE

The present disclosure relates generally to orthopedics and spinal surgery. More specifically, the present disclosure relates to intervertebral prosthetic discs.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is an anterior view of a first embodiment of an intervertebral prosthetic disc;

FIG. 5 is an exploded anterior view of the first embodiment of the intervertebral prosthetic disc;

FIG. 6 is a cross-section view of the first embodiment of the intervertebral prosthetic disc;

FIG. 7 is a lateral view of the first embodiment of the intervertebral prosthetic disc;

FIG. 8 is an exploded lateral view of the first embodiment of the intervertebral prosthetic disc;

FIG. 9 is a plan view of a superior half of the first embodiment of the intervertebral prosthetic disc;

FIG. 10 is a plan view of an inferior half of the first embodiment of the intervertebral prosthetic disc;

FIG. 11 is an exploded lateral view of the first embodiment of the intervertebral prosthetic disc installed within an intervertebral space between a pair of adjacent vertrebrae;

FIG. 12 is an anterior view of the first embodiment of the intervertebral prosthetic disc installed within an intervertebral space between a pair of adjacent vertrebrae;

FIG. 13 is a posterior view of a second embodiment of an intervertebral prosthetic disc;

FIG. 14 is an exploded posterior view of the second embodiment of the intervertebral prosthetic disc;

FIG. 15 is a cross-section view of the second embodiment of the intervertebral prosthetic disc;

FIG. 16 is a lateral view of the second embodiment of the intervertebral prosthetic disc;

FIG. 17 is an exploded lateral view of the second embodiment of the intervertebral prosthetic disc;

FIG. 18 is a plan view of a superior half of the second embodiment of the intervertebral prosthetic disc;

FIG. 19 is another plan view of the superior half of the second embodiment of the intervertebral prosthetic disc;

FIG. 20 is a plan view of an inferior half of the second embodiment of the intervertebral prosthetic disc;

FIG. 21 is another plan view of the inferior half of the second embodiment of the intervertebral prosthetic disc;

FIG. 22 is a lateral view of a third embodiment of an intervertebral prosthetic disc;

FIG. 23 is an exploded lateral view of the third embodiment of the intervertebral prosthetic disc;

FIG. 24 is a cross-section view of the third embodiment of the intervertebral prosthetic disc;

FIG. 25 is a anterior view of the third embodiment of the intervertebral prosthetic disc;

FIG. 26 is a perspective view of a superior component of the third embodiment of the intervertebral prosthetic disc;

FIG. 27 is a perspective view of an inferior component of the third embodiment of the intervertebral prosthetic disc;

FIG. 28 is a lateral view of a fourth embodiment of an intervertebral prosthetic disc;

FIG. 29 is an exploded lateral view of the fourth embodiment of the intervertebral prosthetic disc;

FIG. 30 is a cross-section view of the fourth embodiment of the intervertebral prosthetic disc;

FIG. 31 is a anterior view of the fourth embodiment of the intervertebral prosthetic disc;

FIG. 32 is a perspective view of a superior component of the fourth embodiment of the intervertebral prosthetic disc;

FIG. 33 is a perspective view of an inferior component of the fourth embodiment of the intervertebral prosthetic disc;

FIG. 34 is a posterior view of a fifth embodiment of an intervertebral prosthetic disc;

FIG. 35 is an exploded posterior view of the fifth embodiment of the intervertebral prosthetic disc;

FIG. 36 is a cross-section view of the fifth embodiment of the intervertebral prosthetic disc;

FIG. 37 is a plan view of a superior half of the fifth embodiment of the intervertebral prosthetic disc;

FIG. 38 is a plan view of an inferior half of the fifth embodiment of the intervertebral prosthetic disc;

FIG. 39 is a posterior view of a sixth embodiment of an intervertebral prosthetic disc;

FIG. 40 is an exploded posterior view of the sixth embodiment of the intervertebral prosthetic disc;

FIG. 41 is a cross-section view of the sixth embodiment of the intervertebral prosthetic disc;

FIG. 42 is a plan view of a superior half of the sixth embodiment of the intervertebral prosthetic disc;

FIG. 43 is a plan view of an inferior half of the sixth embodiment of the intervertebral prosthetic disc;

FIG. 44 is a perspective view of a sixth embodiment of an intervertebral prosthetic disc;

FIG. 45 is a superior plan view of the sixth embodiment of the intervertebral prosthetic disc;

FIG. 46 is an anterior plan view of the sixth embodiment of the intervertebral prosthetic disc; and

FIG. 47 is a cross-section view of the sixth embodiment of the intervertebral prosthetic disc taken along line 47-47 in FIG. 45.

### DETAILED DESCRIPTION OF THE DRAWINGS

An intervertebral prosthetic disc is disclosed and can be installed within an intervertebral space between a superior vertebra and an inferior vertebra. The intervertebral prosthetic disc can include an inferior component having a depression formed therein and a superior component having a projection extending therefrom. The projection can be configured to movably engage the depression and allow relative motion between the inferior component and the superior component. Further, the projection can include a superior wear resistant layer configured to engage the depression.

In another embodiment, an intervertebral prosthetic disc is disclosed and can be installed within an intervertebral space between a superior vertebra and an inferior vertebra. The intervertebral prosthetic disc can include an inferior component having a depression formed therein and a superior component having a projection extending therefrom. The projection can include a base and a wear resistant layer disposed on the base. The wear resistant layer can be configured to movably engage the depression and allow relative motion between the inferior component and the superior component.

In yet another embodiment, an intervertebral prosthetic disc is disclosed and can be installed within an intervertebral space between a superior vertebra and an inferior vertebra. The intervertebral prosthetic disc can include an inferior component having an inferior depression formed therein, a superior component having a superior depression formed therein, and a nucleus disposed between the inferior component and the superior component. The nucleus can include a superior wear resistant layer and an inferior wear resistant layer. The superior wear resistant layer of the nucleus can be configured to movably engage the superior depression. Also, the inferior wear resistant layer of the nucleus can be configured to movably engage the inferior depression.

In still another embodiment, an intervertebral prosthetic disc is disclosed and can be installed within an intervertebral space between a superior vertebra and an inferior vertebra. The intervertebral prosthetic disc can include an inferior component having an inferior projection extending therefrom, a superior component having a superior projection extending therefrom, and a nucleus disposed between the inferior component and the superior component. The nucleus can include a superior depression having a superior wear resistant layer therein and an inferior depression having an inferior wear resistant layer therein. The superior wear resistant layer of the nucleus can be configured to movably engage the superior projection. Moreover, the inferior wear resistant layer of the nucleus can be configured to movably engage the inferior projection.

In yet still another embodiment, an intervertebral prosthetic disc is disclosed and can be installed within an intervertebral space between a superior vertebra and an inferior vertebra. The intervertebral prosthetic disc can include an inferior component, a superior component, and a generally toroidal nucleus disposed between the inferior component and the superior component. The nucleus can include a core and an outer wear resistant layer disposed on the core. The outer wear resistant layer of the core can be configured to movably engage the inferior component and the superior component.

Description of Relevant Anatomy

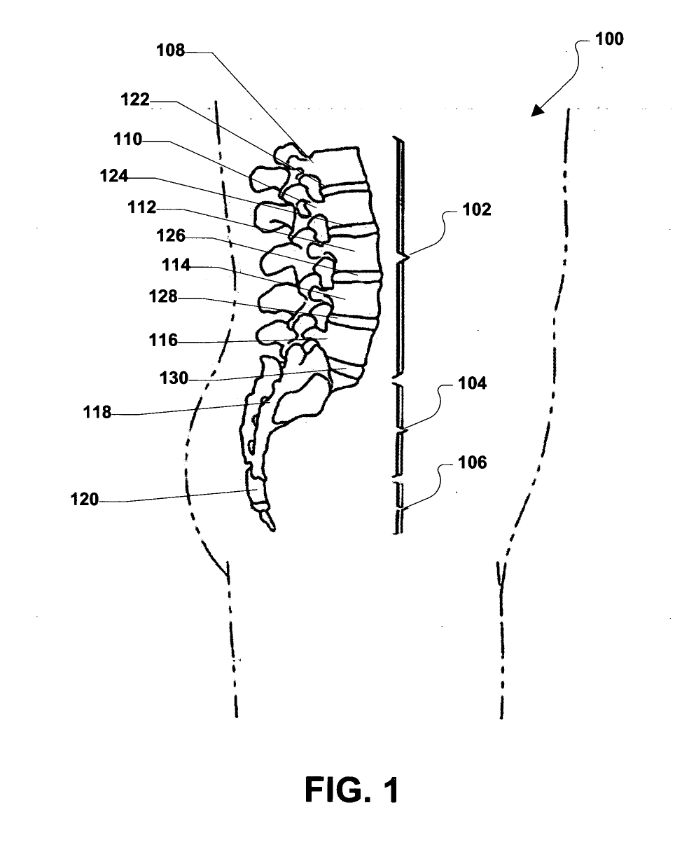

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

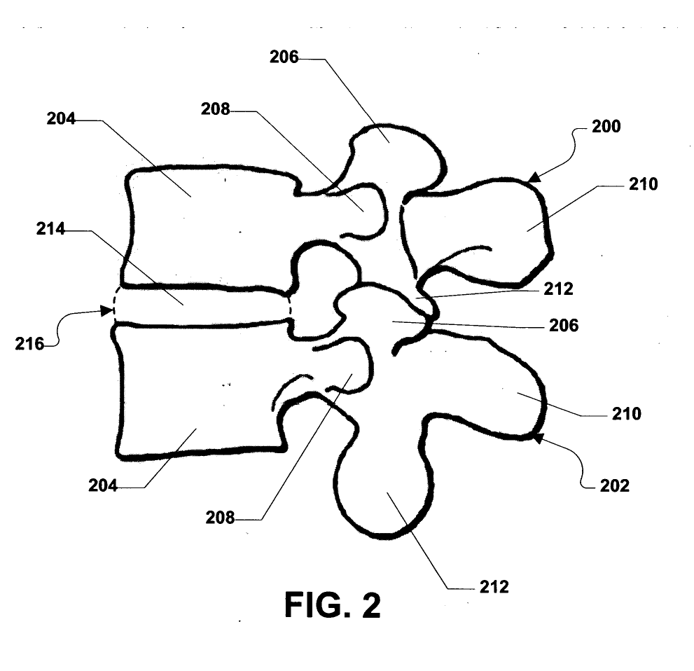

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

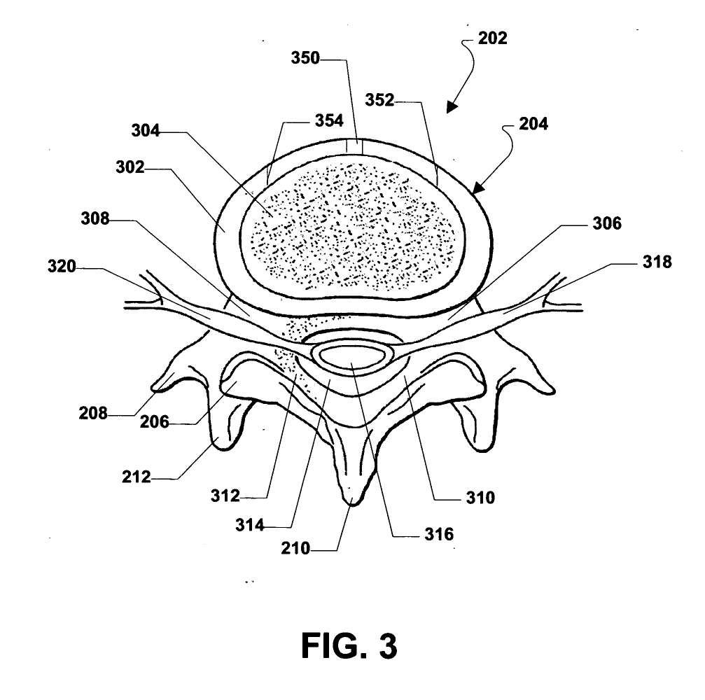

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel cutting device, e.g., a keel chisel designed to cut a groove in a vertebra, prior to the installation of the intervertebral prosthetic disc. Further, the keel groove 350 is sized and shaped to receive and engage a keel, described in detail below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

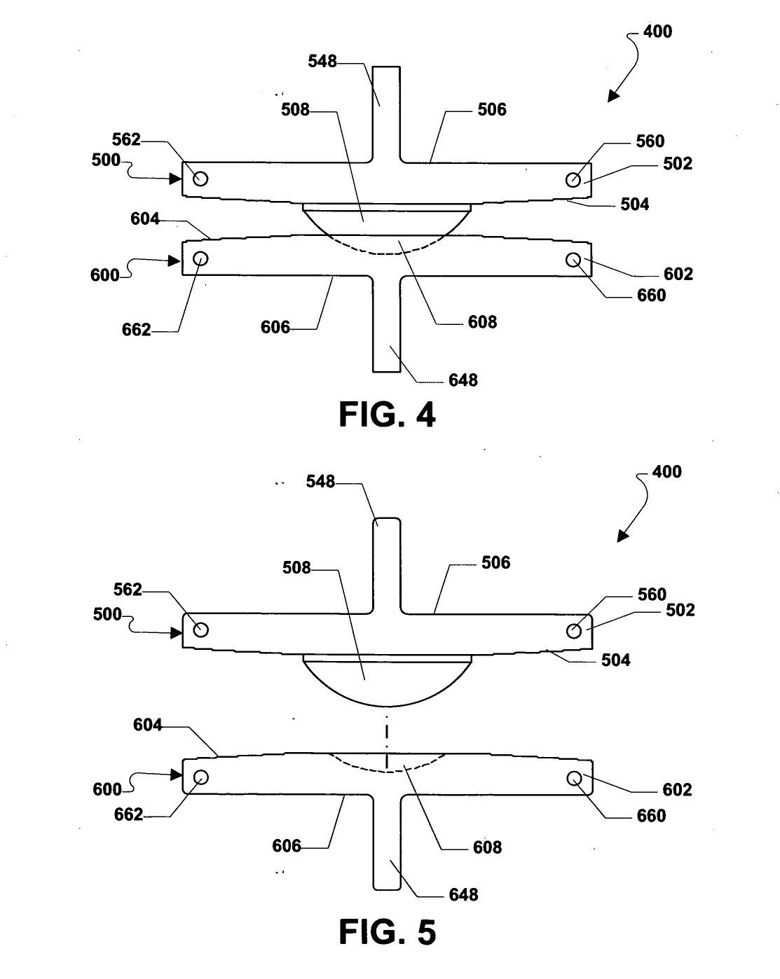

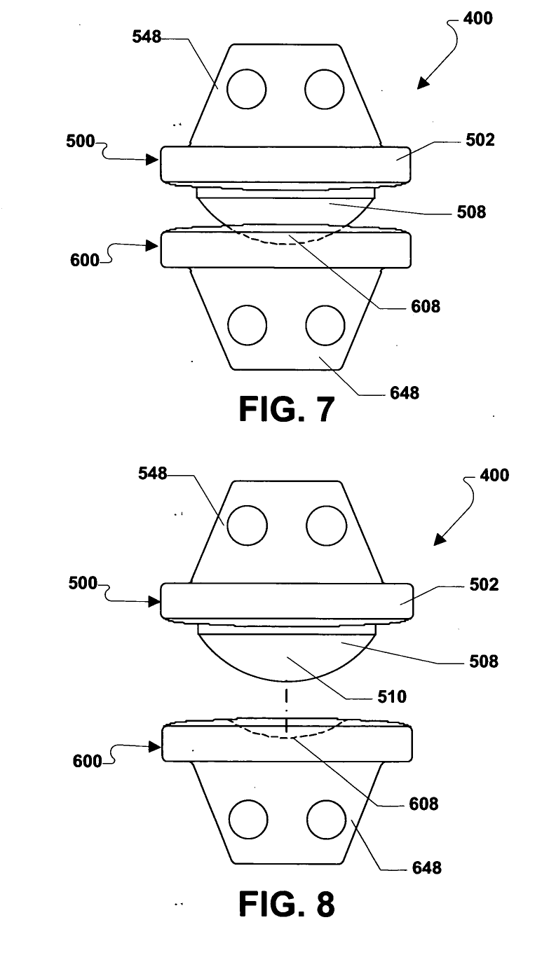

Description of a First Embodiment of an Intervertebral Prosthetic Disc Referring to FIGS. 4 through 10 a first embodiment of an intervertebral prosthetic disc is shown and is generally designated 400. As illustrated, the intervertebral prosthetic disc 400 can include a superior component 500 and an inferior component 600. In a particular embodiment, the components 500, 600 can be made from one or more biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 500, 600 can be made from any other substantially rigid biocompatible materials.

In a particular embodiment, the superior component 500 can include a superior support plate 502 that has a superior articular surface 504 and a superior bearing surface 506. In a particular embodiment, the superior articular surface 504 can be generally curved and the superior bearing surface 506 can be substantially flat. In an alternative embodiment, the superior articular surface 504 can be substantially flat and at least a portion of the superior bearing surface 506 can be generally curved.

As illustrated in FIG. 4 through FIG. 8, a projection 508 extends from the superior articular surface 504 of the superior support plate 502. In a particular embodiment, the projection 508 has a hemi-spherical shape. Alternatively, the projection 508 can have an elliptical shape, a cylindrical shape, or other arcuate shape.

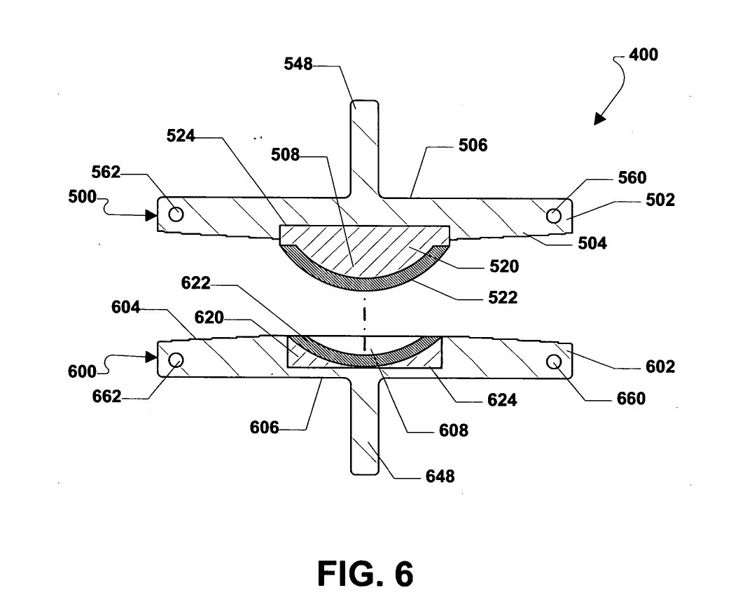

Referring to FIG. 6, the projection 508 can include a base 520 and a superior wear resistant layer 522 affixed to, deposited on, or otherwise disposed on, the base 520. In a particular embodiment, the base 520 can act as a substrate and the superior wear resistant layer 522 can be deposited on the base 520. Further, the base 520 can engage a cavity 524 that can be formed in the superior support plate 502. In a particular embodiment, the cavity 524 can be sized and shaped to receive the base 520 of the projection 508. Further, the base 520 of the projection 508 can be press fit into the cavity 524.

In a particular embodiment, the base 520 of the projection 508 can be made from or at least include an inorganic, carbon-based substance, such as graphite, suitable for receiving the wear resistant layer thereon. Further, in a particular embodiment, the superior wear resistant layer 522 can be formed of or at least include pyrolytic carbon that is deposited on the base 520. In one embodiment, pyrolytic carbon can be deposited on a suitable substrate via chemical vapor deposition at a temperature between one thousand degrees Kelvin and two thousand five hundred degrees Kelvin (1000° K-2500° K).

As such, the base 520 can be made from a material that can allow pyrolytic carbon to be deposited thereon in a manner such that the deposited pyrolytic carbon can withstand multiple articulation cycles without substantial detachment. The base 520 can be fitted into a superior support plate 502 made from one or more of the materials described herein. Accordingly, the superior support plate 502 may be made from a material that does not adequately facilitate the deposition of pyrolytic carbon thereon.

Also, in a particular embodiment, the base 520 can be roughened prior to the deposition of the pyrolytic carbon thereon. For example, the base 520 can be roughened using a roughening process. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 520 on which the pyrolytic carbon is deposited can be serrated and can include one or more teeth, spikes, or other protrusions extending therefrom. The serrations of the base 520 can facilitate anchoring of the pyrolytic carbon on the base 520 and can substantially reduce the likelihood of delamination of the superior wear resistant layer 522 from the base 520.

In a particular embodiment, the superior wear resistant layer 522 can have a thickness in a range of fifty micrometers to five millimeters (50 μm-5 mm). Further, the superior wear resistant layer 522 can have a thickness in a range of two hundred micrometers to two millimeters (200 μm-2 mm). In a particular embodiment, the serrations that can be formed on the surface of the base 520 can have a height that is at most half of the thickness of the superior wear resistant layer 522. Accordingly, the likelihood that the serrations will protrude through the superior wear resistant layer 522 is substantially minimized.

Additionally, in a particular embodiment, a Young's modulus of the superior wear resistant layer 522 can be substantially greater than a Young's modulus of the base 520. Also, a hardness of the superior wear resistant layer 522 can be substantially greater than a hardness of the base 520. Further, a toughness of the superior wear resistant layer 522 can be substantially greater than a toughness of the base 520. In a particular embodiment, the superior wear resistant layer 522 can be annealed immediately after deposition in order to minimize cracking of the superior wear resistant layer. Also, the superior wear resistant layer 522 can be polished in order to minimize surface irregularities of the superior wear resistant layer 522 and increase a smoothness of the superior wear resistant layer 522.

FIG. 4 through FIG. 8 indicate that the superior component 500 can include a superior keel 548 that extends from superior bearing surface 506. During installation, described below, the superior keel 548 can at least partially engage a keel groove that can be established within a cortical rim of a vertebra. Further, the superior keel 548 can be coated with a bone-growth promoting substance, e.g., a hydroxyapatite coating formed of calcium phosphate. Additionally, the superior bearing surface 506 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

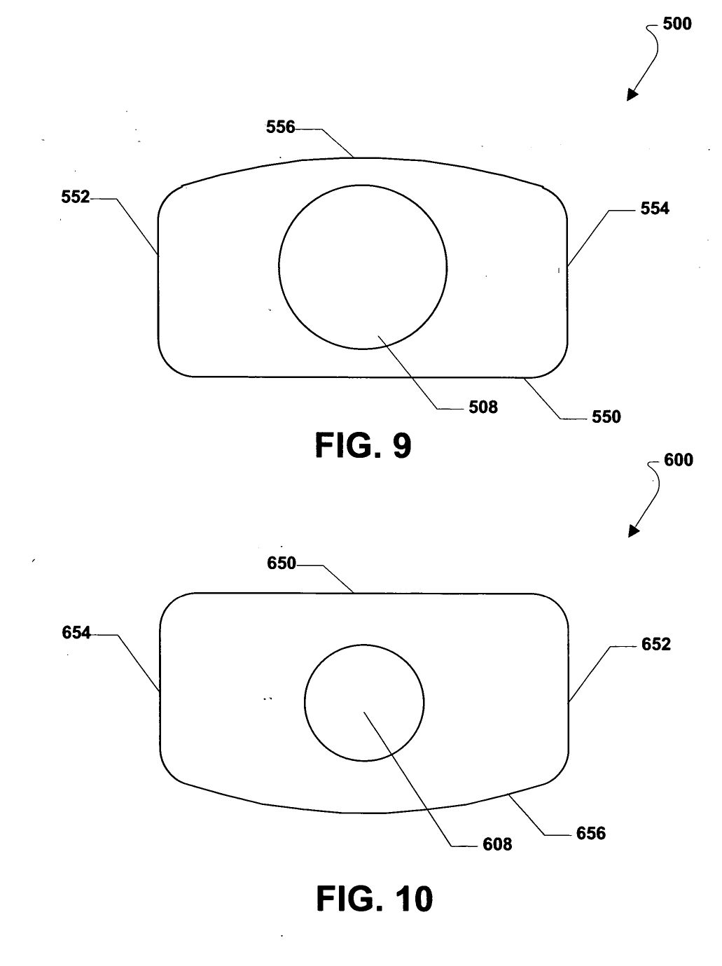

As illustrated in FIG. 9, the superior component 500 can be generally rectangular in shape. For example, the superior component 500 can have a substantially straight posterior side 550. A first straight lateral side 552 and a second substantially straight lateral side 554 can extend substantially perpendicular from the posterior side 550 to an anterior side 556. In a particular embodiment, the anterior side 556 can curve outward such that the superior component 500 is wider through the middle than along the lateral sides 552, 554. Further, in a particular embodiment, the lateral sides 552, 554 are substantially the same length.

FIG. 4 through FIG. 6 show that the superior component 500 can include a first implant inserter engagement hole 560 and a second implant inserter engagement hole 562. In a particular embodiment, the implant inserter engagement holes 560, 562 are configured to receive respective dowels, or pins, that extend from an implant inserter (not shown) that can be used to facilitate the proper installation of an intervertebral prosthetic disc, e.g., the intervertebral prosthetic disc 400 shown in FIG. 4 through FIG. 10.

In a particular embodiment, the inferior component 600 can include an inferior support plate 602 that has an inferior articular surface 604 and an inferior bearing surface 606. In a particular embodiment, the inferior articular surface 604 can be generally curved and the inferior bearing surface 606 can be substantially flat. In an alternative embodiment, the inferior articular surface 604 can be substantially flat and at least a portion of the inferior bearing surface 606 can be generally curved.

As illustrated in FIG. 4 through FIG. 8, a depression 608 extends into the inferior articular surface 604 of the inferior support plate 602. In a particular embodiment, the depression 608 is sized and shaped to receive the projection 508 of the superior component 500. For example, the depression 608 can have a hemi-spherical shape. Alternatively, the depression 608 can have an elliptical shape, a cylindrical shape, or other arcuate shape.

Referring to FIG. 6, the depression 608 can include a base 620 and an inferior wear resistant layer 622 affixed to, deposited on, or otherwise disposed on, the base 620. In a particular embodiment, the base 620 can act as a substrate and the inferior wear resistant layer 622 can be deposited on the base 620. Further, the base 620 can engage a cavity 624 that can be formed in the inferior support plate 602. In a particular embodiment, the cavity 624 can be sized and shaped to receive the base 620 of the depression 608. Further, the base 620 of the depression 608 can be press fit into the cavity 624.

In a particular embodiment, the base 620 of the depression 608 can be made from or at least include an inorganic, carbon-based substance, such as graphite, suitable for receiving the wear resistant layer thereon. Further, in a particular embodiment, the inferior wear resistant layer 622 can be formed of or at least include pyrolytic carbon that is deposited on the base 620. In one embodiment, pyrolytic carbon can be deposited on a suitable substrate via chemical vapor deposition at a temperature between one thousand degrees Kelvin and two thousand five hundred degrees Kelvin (1000° K.-2500° K).

As such, the base 620 can be made from a material that can allow pyrolytic carbon to be deposited thereon in a manner such that the deposited Pyrolytic carbon can withstand multiple articulation cycles without substantial detachment. The base 620 can be fitted into an inferior support plate 602 made from one or more of the materials described herein. Accordingly, the inferior support plate 602 may be made from a material that does not adequately facilitate the deposition of pyrolytic carbon thereon.

Also, in a particular embodiment, the base 620 can be roughened prior to the deposition of the pyrolytic carbon thereon. For example, the base 620 can be roughened using a roughening process. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 620 on which the pyrolytic-carbon is deposited can be serrated and can include one or more teeth, spikes, or other protrusions extending therefrom. The serrations of the base 620 can facilitate anchoring of the pyrolytic carbon on the base 620 and can substantially reduce the likelihood of delamination of the inferior wear resistant layer 622 from the base 620.

In a particular embodiment, the inferior wear resistant layer 622 can have a thickness in a range of fifty micrometers to five millimeters (50 μm-5 mm). Further, the inferior wear resistant layer 622 can have a thickness in a range of two hundred micrometers to two millimeters (200 μm-2 mm). In a particular embodiment, the serrations that can be formed on the surface of the base 620 can have a height that is at most half of the thickness of the inferior wear resistant layer 622. Accordingly, the likelihood that the serrations will protrude through the inferior wear resistant layer 622 is substantially minimized.

Additionally, in a particular embodiment, a Young's modulus of the inferior wear resistant layer 622 can be substantially greater than a Young's modulus of the base 620. Also, a hardness of the inferior wear resistant layer 622 can be substantially greater than a hardness of the base 620. Further, a toughness of the inferior wear resistant layer 622 can be substantially greater than a toughness of the base 620. In a particular embodiment, the inferior wear resistant layer 622 can be annealed immediately after deposition in order to minimize cracking of the inferior wear resistant layer. Also, the inferior wear resistant layer 622 can be polished in order to minimize surface irregularities of the inferior wear resistant layer 622 and increase a smoothness of the inferior wear resistant layer 622.

FIG. 4 through FIG. 8 indicate that the inferior component 600 can include an inferior keel 648 that extends from inferior bearing surface 606. During installation, described below, the inferior keel 648 can at least partially engage a keel groove that can be established within a cortical rim of a vertebra, e.g., the keel groove 350 shown in FIG. 3. Further, the inferior keel 648 can be coated with a bone-growth promoting substance, e.g., a hydroxyapatite coating formed of calcium phosphate. Additionally, the inferior bearing surface 606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

In a particular embodiment, as shown in FIG. 10, the inferior component 600 can be shaped to match the shape of the superior component 500, shown in FIG. 9. Further, the inferior component 600 can be generally rectangular in shape. For example, the inferior component 600 can have a substantially straight posterior side 650. A first straight lateral side 652 and a second substantially straight lateral side 654 can extend substantially perpendicular from the posterior side 650 to an anterior side 656. In a particular embodiment, the anterior side 656 can curve outward such that the inferior component 600 is wider through the middle than along the lateral sides 652, 654. Further, in a particular embodiment, the lateral sides 652, 654 are substantially the same length.

FIG. 4 through FIG. 6 show that the inferior component 600 can include a first implant inserter engagement hole 660 and a second implant inserter engagement hole 662. In a particular embodiment, the implant inserter engagement holes 660, 662 are configured to receive respective dowels, or pins, that extend from an implant inserter (not shown) that can be used to facilitate the proper installation of an intervertebral prosthetic disc, e.g., the intervertebral prosthetic disc 400 shown in FIG. 4 through FIG. 10.

In a particular embodiment, the overall height of the intervertebral prosthetic device 400 can be in a range from fourteen millimeters to forty-six millimeters (14-46 mm). Further, the installed height of the intervertebral prosthetic device 400 can be in a range from eight millimeters to sixteen millimeters (8-16 mm). In a particular embodiment, the installed height can be substantially equivalent to the distance between an inferior vertebra and a superior vertebra when the intervertebral prosthetic device 400 is installed there between.

In a particular embodiment, the length of the intervertebral prosthetic device 400, e.g., along a longitudinal axis, can be in a range from thirty millimeters to forty millimeters (30-40 mm). Additionally, the width of the intervertebral prosthetic device 400, e.g., along a lateral axis, can be in a range from twenty-five millimeters to forty millimeters (25-40 mm). Moreover, in a particular embodiment, each keel 548, 648 can have a height in a range from three millimeters to fifteen millimeters (3-15 mm).

### INSTALLATION OF THE FIRST EMBODIMENT WITHIN AN INTERVERTEBRAL SPACE

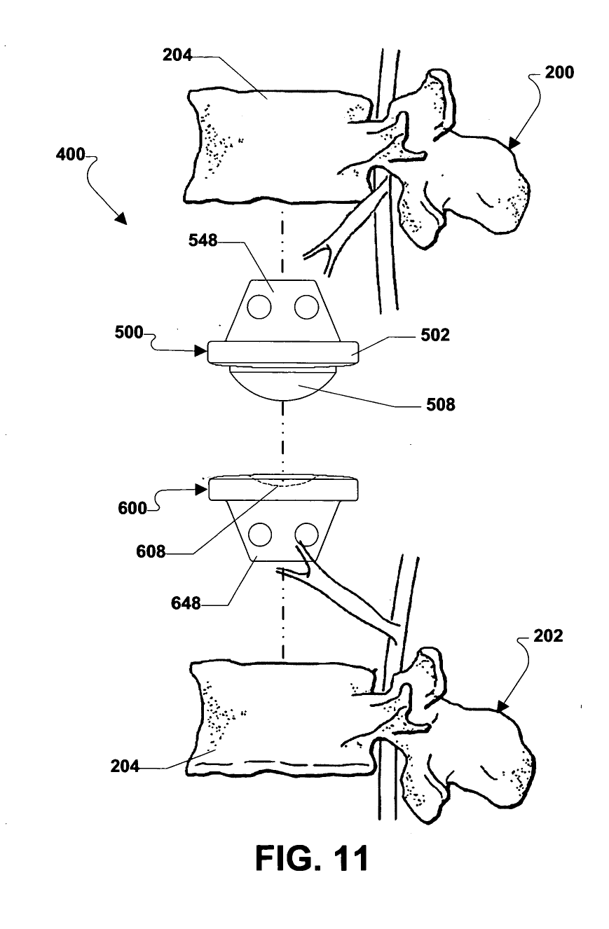

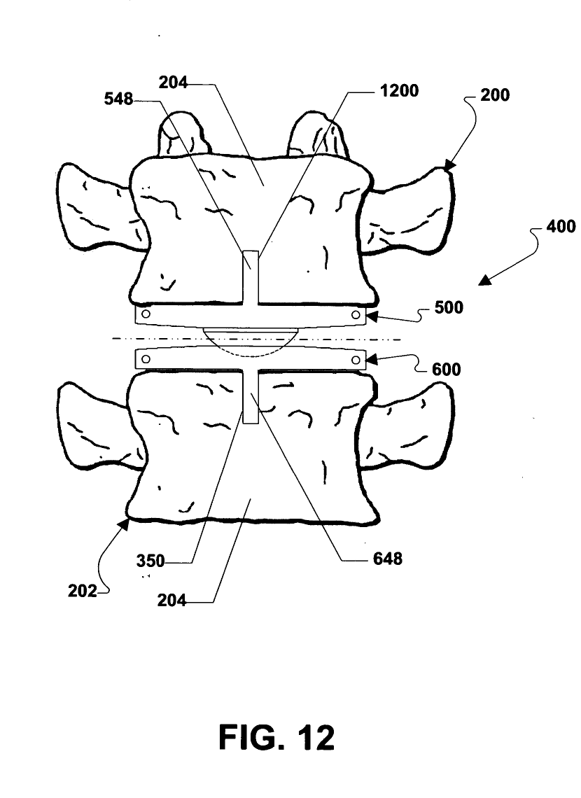

Referring to FIG. 11 and FIG. 12, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 400 described in conjunction with FIG. 4 through FIG. 10. Alternatively, the intervertebral prosthetic disc can be an intervertebral prosthetic disc according to any of the embodiments disclosed herein.

As shown in FIG. 11 and FIG. 12, the intervertebral prosthetic disc 400 is installed within the intervertebral space 214 that can be established between the superior vertebra 200 and the inferior vertebra 202 by removing vertebral disc material (not shown). FIG. 12 shows that the superior keel 548 of the superior component 500 can at least partially engage the cancellous bone and cortical rim of the superior vertebra 200. Further, as shown in FIG. 12, the superior keel 548 of the superior component 500 can at least partially engage a superior keel groove 1200 that can be established within the vertebral body 204 of the superior vertebra 202. In a particular embodiment, the vertebral body 204 can be further cut to allow the superior support plate 502 of the superior component 500 to be at least partially recessed into the vertebral body 204 of the superior vertebra 200.

Also, as shown in FIG. 11, the inferior keel 648 of the inferior component 600 can at least partially engage the cancellous bone and cortical rim of the inferior vertebra 202. Further, as shown in FIG. 12, the inferior keel 648 of the inferior component 600 can at least partially engage the inferior keel groove 350, previously introduced and described in conjunction with FIG. 3, which can be established within the vertebral body 204 of the inferior vertebra 202. In a particular embodiment, the vertebral body 204 can be further cut to allow the inferior support plate 602 of the inferior component 600 to be at least partially recessed into the vertebral body 204 of the inferior vertebra 200.

As illustrated in FIG. 11 and FIG. 12, the projection 508 that extends from the superior component 500 of the intervertebral prosthetic disc 400 can at least partially engage the depression 608 that is formed within the inferior component 600 of the intervertebral prosthetic disc 400. More specifically, the superior wear resistant layer 522 of the superior component 500 can at least partially engage the inferior wear resistant layer 622 of the inferior component 600. Further, the superior wear resistant layer 522 of the superior component 500 can movably engage the inferior wear resistant layer 622 of the inferior component 600 to allow relative motion between the superior component 500 and the inferior component 600.

It is to be appreciated that when the intervertebral prosthetic disc 400 is installed between the superior vertebra 200 and the inferior vertebra 202, the intervertebral prosthetic disc 400 allows relative motion between the superior vertebra 200 and the inferior vertebra 202. Specifically, the configuration of the superior component 500 and the inferior component 600 allows the superior component 500 to rotate with respect to the inferior component 600. As such, the superior vertebra 200 can rotate with respect to the inferior vertebra 202.

In a particular embodiment, the intervertebral prosthetic disc 400 can allow angular movement in any radial direction relative to the intervertebral prosthetic disc 400.

Further, as depicted in FIG. 10 through 12, the inferior component 600 can be placed on the inferior vertebra 202 so that the center of rotation of the inferior component 600 is substantially aligned with the center of rotation of the inferior vertebra 202. Similarly, the superior component 500 can be placed relative to the superior vertebra 200 so that the center of rotation of the superior component 500 is substantially aligned with the center of rotation of the superior vertebra 200. Accordingly, when the vertebral disc, between the inferior vertebra 202 and the superior vertebra 200, is removed and replaced with the intervertebral prosthetic disc 400 the relative motion of the vertebrae 200, 202 provided by the vertebral disc is substantially replicated.

### DESCRIPTION OF A SECOND EMBODIMENT OF AN INTERVERTEBRAL PROSTHETIC DISC

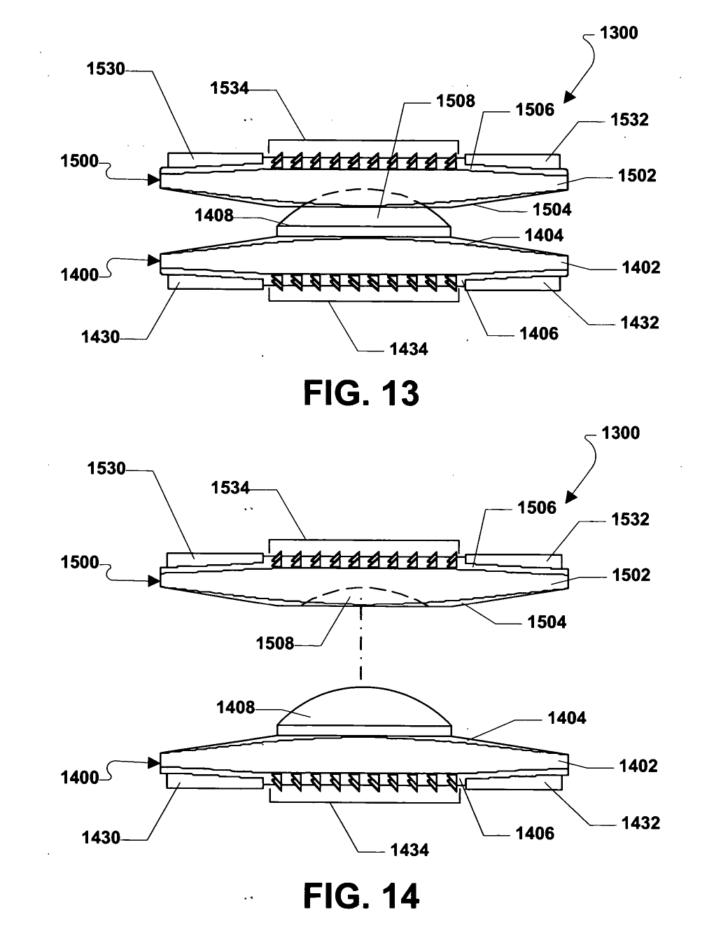

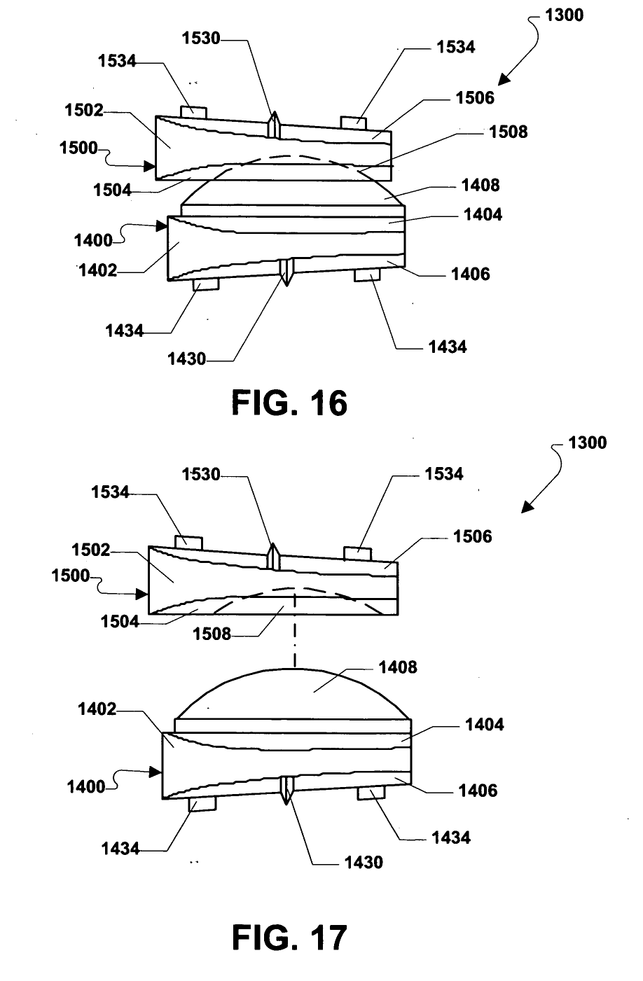

Referring to FIGS. 13 through 21 a second embodiment of an intervertebral prosthetic disc is shown and is generally designated 1300. As illustrated, the intervertebral prosthetic disc 1300 can include an inferior component 1400 and a superior component 1500. In a particular embodiment, the components 1400, 1500 can be made from one or more biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 1400, 1500 can be made from any other substantially rigid biocompatible materials.

In a particular embodiment, the inferior component 1400 can include an inferior support plate 1402 that has an inferior articular surface 1404 and an inferior bearing surface 1406. In a particular embodiment, the inferior articular surface 1404 can be generally rounded and the inferior bearing surface 1406 can be generally flat.

As illustrated in FIG. 13 through FIG. 21, a projection 1408 extends from the inferior articular surface 1404 of the inferior support plate 1402. In a particular embodiment, the projection 1408 has a hemi-spherical shape. Alternatively, the projection 1408 can have an elliptical shape, a cylindrical shape, or other arcuate shape.

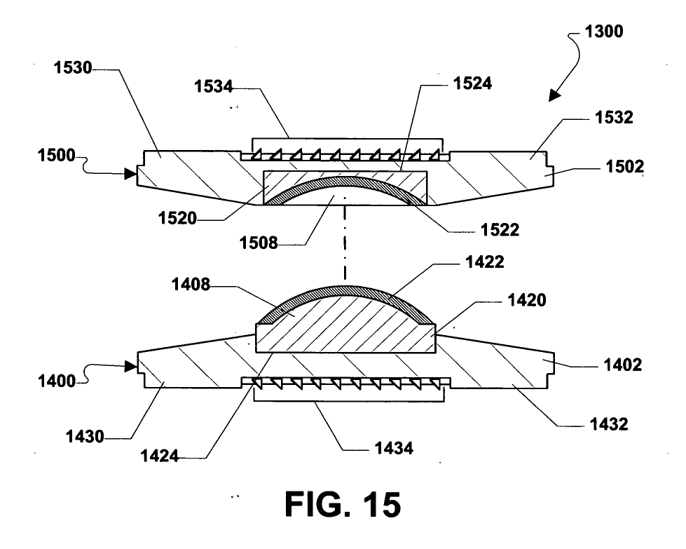

Referring to FIG. 15, the projection 1408 can include a base 1420 and an inferior wear resistant layer 1422 affixed to, deposited on, or otherwise disposed on, the base 1420. In a particular embodiment, the base 1420 can act as a substrate and the inferior wear resistant layer 1422 can be deposited on the base 1420. Further, the base 1420 can engage a cavity 1424 that can be formed in the inferior support plate 1402. In a particular embodiment, the cavity 1424 can be sized and shaped to receive the base 1420 of the projection 1408. Further, the base 1420 of the projection 1408 can be press fit into the cavity 1424.

In a particular embodiment, the base 1420 of the projection can be made from or at least include an inorganic, carbon-based substance, such as graphite, suitable for receiving the wear resistant layer thereon. Further, in a particular embodiment, the inferior wear resistant layer 1422 can be formed of or at least include pyrolytic carbon that is deposited on the base 1420. In one embodiment, pyrolytic carbon can be deposited on a suitable substrate via chemical vapor deposition at a temperature between one thousand degrees Kelvin and two thousand five hundred degrees Kelvin (1000° K-2500° K).

As such, the base 1420 can be made from a material that can allow pyrolytic carbon to be deposited thereon in a manner such that the deposited pyrolytic carbon can withstand multiple articulation cycles without substantial detachment. The base 1420 can be fitted into an inferior support plate 1402 made from one or more of the materials described herein. Accordingly, the inferior support plate 1402 may be made from a material that does not adequately facilitate the deposition of pyrolytic carbon thereon.

Also, in a particular embodiment, the base 1420 can be roughened prior to the deposition of the pyrolytic carbon thereon. For example, the base 1420 can be roughened using a roughening process. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 1420 on which the pyrolytic carbon is deposited can be serrated and can include one or more teeth, spikes, or other protrusions extending therefrom. The serrations of the base 1420 can facilitate anchoring of the pyrolytic carbon on the base 1420 and can substantially reduce the likelihood of delamination of the inferior wear resistant layer 1422 from the base 1420.

In a particular embodiment, the inferior wear resistant layer 1422 can have a thickness in a range of fifty micrometers to five millimeters (50 μm-5 mm). Further, the inferior wear resistant layer 1422 can have a thickness in a range of two hundred micrometers to two millimeters (200 μm-2 mm). In a particular embodiment, the serrations that can be formed on the surface of the base 1420 can have a height that is at most half of the thickness of the inferior wear resistant layer 1422. Accordingly, the likelihood that the serrations will protrude through the inferior wear resistant layer 1422 is substantially minimized.

Additionally, in a particular embodiment, a Young's modulus of the inferior wear resistant layer 1422 can be substantially greater than a Young's modulus of the base 1420. Also, a hardness of the inferior wear resistant layer 1422 can be substantially greater than a hardness of the base 1420. Further, a toughness of the inferior wear resistant layer 1422 can be substantially greater than a toughness of the base 1420. In a particular embodiment, the inferior wear resistant layer 1422 can be annealed immediately after deposition in order to minimize cracking of the inferior wear resistant layer. Also, the inferior wear resistant layer 1422 can be polished in order to minimize surface irregularities of the inferior wear resistant layer 1422 and increase a smoothness of the inferior wear resistant layer 1422.

FIG. 13 through FIG. 17 and FIG. 19 also show that the inferior component 1400 can include a first inferior keel 1430, a second inferior keel 1432, and a plurality of inferior teeth 1434 that extend from the inferior bearing surface 1406. As shown, in a particular embodiment, the inferior keels 1430, 1432 and the inferior teeth 1434 are generally saw-tooth, or triangle, shaped. Further, the inferior keels 1430, 1432 and the inferior teeth 1434 are designed to engage cancellous bone, cortical bone, or a combination thereof of an inferior vertebra. Additionally, the inferior teeth 1434 can prevent the inferior component 1400 from moving with respect to an inferior vertebra after the intervertebral prosthetic disc 1300 is installed within the intervertebral space between the inferior vertebra and the superior vertebra.

In a particular embodiment, the inferior teeth 1434 can include other projections such as spikes, pins, blades, or a combination thereof that have any cross-sectional geometry.

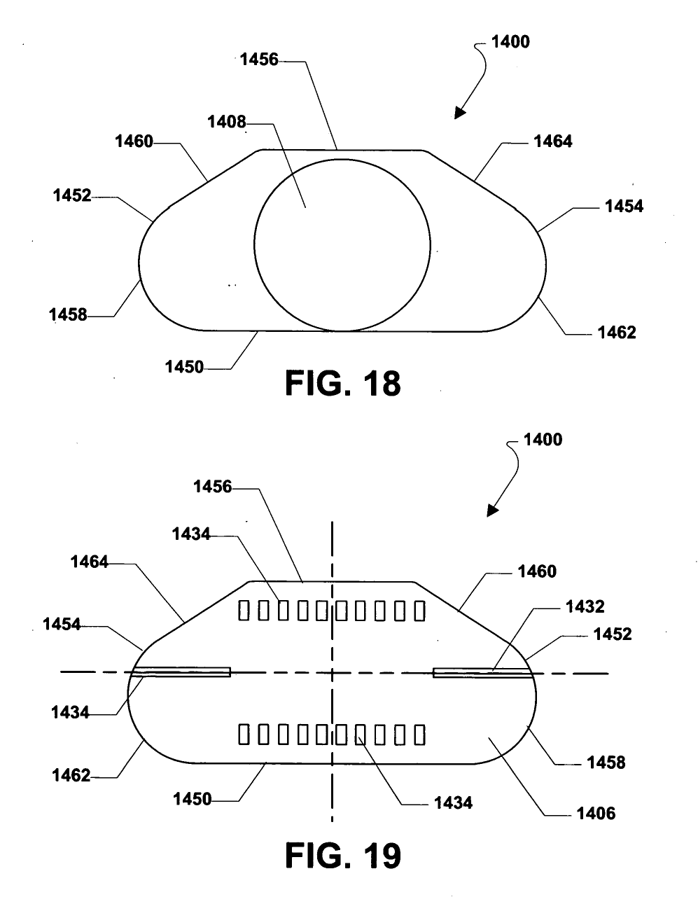

As illustrated in FIG. 18 and FIG. 19, the inferior component 1400 can be generally shaped to match the general shape of the vertebral body of a vertebra. For example, the inferior component 1400 can have a general trapezoid shape and the inferior component 1400 can include a posterior side 1450. A first lateral side 1452 and a second lateral side 1454 can extend from the posterior side 1450 to an anterior side 1456. In a particular embodiment, the first lateral side 1452 can include a curved portion 1458 and a straight portion 1460 that extends at an angle toward the anterior side 1456. Further, the second lateral side 1454 can also include a curved portion 1462 and a straight portion 1464 that extends at an angle toward the anterior side 1456.

As shown in FIG. 18 and FIG. 19, the anterior side 1456 of the inferior component 1400 can be relatively shorter than the posterior side 1450 of the inferior component 1400. Further, in a particular embodiment, the anterior side 1456 is substantially parallel to the posterior side 1450. As indicated in FIG. 18, the projection 1408 can be situated relative to the inferior articular surface 1404 such that the perimeter of the projection 1408 is tangential to the posterior side 1450 of the inferior component 1400. In alternative embodiments (not shown), the projection 1408 can be situated relative to the inferior articular surface 1404 such that the perimeter of the projection 1408 is tangential to the anterior side 1456 of the inferior component 1400 or tangential to both the anterior side 1456 and the posterior side 1450.

In a particular embodiment, the superior component 1500 can include a superior support plate 1502 that has a superior articular surface 1504 and a superior bearing surface 1506. In a particular embodiment, the superior articular surface 1504 can be generally rounded and the superior bearing surface 1506 can be generally flat.

As illustrated in FIG. 13 through FIG. 21, a depression 1508 extends into the superior articular surface 1504 of the superior support plate 1502. In a particular embodiment, the depression 1508 has a hemi-spherical shape. Alternatively, the depression 1508 can have an elliptical shape, a cylindrical shape, or other arcuate shape.

Referring to FIG. 15, the depression 1508 can include a base 1520 and a superior wear resistant layer 1522 affixed to, deposited on, or otherwise disposed on, the base 1520. In a particular embodiment, the base 1520 can act as a substrate and the superior wear resistant layer 1522 can be deposited on the base 1520. Further, the base 1520 can engage a cavity 1524 that can be formed in the superior support plate 1502. In a particular embodiment, the cavity 1524 can be sized and shaped to receive the base 1520 of the depression 1508. Further, the base 1520 of the depression 1508 can be press fit into the cavity 1524.

In a particular embodiment, the base 1520 of the depression 1508 can be made from or at least include an inorganic, carbon-based substance, such as graphite, suitable for receiving the wear resistant layer thereon. Further, in a particular embodiment, the superior wear resistant layer 1522 can be formed of or at least include pyrolytic carbon that is deposited on the base 1520. In one embodiment, pyrolytic carbon can be deposited on a suitable substrate via chemical vapor deposition at a temperature between one thousand degrees Kelvin and two thousand five hundred degrees Kelvin (1000° K-2500° K).

As such, the base 1520 can be made from a material that can allow pyrolytic carbon to be deposited thereon in a manner such that the deposited pyrolytic carbon can withstand multiple articulation cycles without substantial detachment. The base 1520 can be fitted into a superior support plate 1502 made from one or more of the materials described herein. Accordingly, the superior support plate 1502 may be made from a material that does not adequately facilitate the deposition of pyrolytic carbon thereon.

Also, in a particular embodiment, the base 1520 can be roughened prior to the deposition of the pyrolytic carbon thereon. For example, the base 1520 can be roughened using a roughening process. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 1520 on which the pyrolytic carbon is deposited can be serrated and can include one or more teeth, spikes, or other protrusions extending therefrom. The serrations of the base 1520 can facilitate anchoring of the pyrolytic carbon on the base 1520 and can substantially reduce the likelihood of delamination of the superior wear resistant layer 1522 from the base 1520.

In a particular embodiment, the superior wear resistant layer 1522 can have a thickness in a range of fifty micrometers to five millimeters (50 μm-5 mm). Further, the superior wear resistant layer 1522 can have a thickness in a range of two hundred micrometers to two millimeters (200 μm-2 mm). In a particular embodiment, the serrations that can be formed on the surface of the base 1520 can have a height that is at most half of the thickness of the superior wear resistant layer 1522. Accordingly, the likelihood that the serrations will protrude through the superior wear resistant layer 1522 is substantially minimized.

Additionally, in a particular embodiment, a Young's modulus of the superior wear resistant layer 1522 can be substantially greater than a Young's modulus of the base 1520. Also, a hardness of the superior wear resistant layer 1522 can be substantially greater than a hardness of the base 1520. Further, a toughness of the superior wear resistant layer 1522 can be substantially greater than a toughness of the base 1520. In a particular embodiment, the superior wear resistant layer 1522 can be annealed immediately after deposition in order to minimize cracking of the superior wear resistant layer. Also, the superior wear resistant layer 1522 can be polished in order to minimize surface irregularities of the superior wear resistant layer 1522 and increase a smoothness of the superior wear resistant layer 1522.

FIG. 13 through FIG. 11 and FIG. 21 also show that the superior component 1500 can include a first superior keel 1530, a second superior keel 1532, and a plurality of superior teeth 1534 that extend from the superior bearing surface 1506. As shown, in a particular embodiment, the superior keels 1530, 1532 and the superior teeth 1534 are generally saw-tooth, or triangle, shaped. Further, the superior keels 1530, 1532 and the superior teeth 1534 are designed to engage cancellous bone, cortical bone, or a combination thereof, of a superior vertebra. Additionally, the superior teeth 1534 can prevent the superior component 1500 from moving with respect to a superior vertebra after the intervertebral prosthetic disc 1300 is installed within the intervertebral space between the inferior vertebra and the superior vertebra.

In a particular embodiment, the superior teeth 1534 can include other depressions such as spikes, pins, blades, or a combination thereof that have any cross-sectional geometry.

In a particular embodiment, the superior component 1500 can be shaped to match the shape of the inferior component 1400, shown in FIG. 18 and FIG. 19. Further, the superior component 1500 can be shaped to match the general shape of a vertebral body of a vertebra. For example, the superior component 1500 can have a general trapezoid shape and the superior component 1500 can include a posterior side 1550. A first lateral side 1552 and a second lateral side 1554 can extend from the posterior side 1550 to an anterior side 1556. In a particular embodiment, the first lateral side 1552 can include a curved portion 1558 and a straight portion 1560 that extends at an angle toward the anterior side 1556. Further, the second lateral side 1554 can also include a curved portion 1562 and a straight portion 1564 that extends at an angle toward the anterior side 1556.

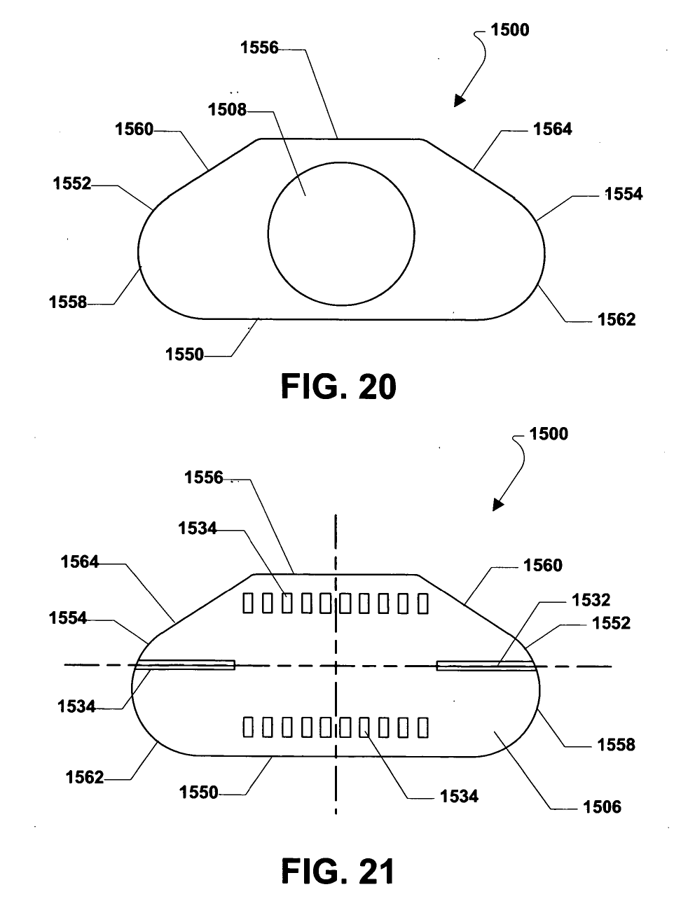

As shown in FIG. 20 and FIG. 21, the anterior side 1556 of the superior component 1500 can be relatively shorter than the posterior side 1550 of the superior component 1500. Further, in a particular embodiment, the anterior side 1556 is substantially parallel to the posterior side 1550.

In a particular embodiment, the overall height of the intervertebral prosthetic device 1300 can be in a range from six millimeters to twenty-two millimeters (6-22 mm). Further, the installed height of the intervertebral prosthetic device 1300 can be in a range from four millimeters to sixteen millimeters (4-15 mm). In a particular embodiment, the installed height can be substantially equivalent to the distance between an inferior vertebra and a superior vertebra when the intervertebral prosthetic device 1300 is installed there between.

In a particular embodiment, the length of the intervertebral prosthetic device 1300, e.g., along a longitudinal axis, can be in a range from thirty-three millimeters to fifty millimeters (33-50 mm). Additionally, the width of the intervertebral prosthetic device 1300, e.g., along a lateral axis, can be in a range from eighteen millimeters to twenty-nine millimeters (18-29 mm).

In a particular embodiment, the intervertebral prosthetic disc 1300 can be considered to be “low profile.” The low profile the intervertebral prosthetic device 1300 can allow the intervertebral prosthetic device 1300 to be implanted into an intervertebral space between an inferior vertebra and a superior vertebra laterally through a patient's psoas muscle, e.g., through an insertion device. Accordingly, the risk of damage to a patient's spinal cord or sympathetic chain can be substantially minimized. In alternative embodiments, all of the superior and inferior teeth 1418, 1518 can be oriented to engage in a direction substantially opposite the direction of insertion of the prosthetic disc into the intervertebral space.

Further, the intervertebral prosthetic disc 1300 can have a general “bullet” shape as shown in the posterior plan view, described herein. The bullet shape of the intervertebral prosthetic disc 1300 can further allow the intervertebral prosthetic disc 1300 to be inserted through the patient's psoas muscle while minimizing risk to the patient's spinal cord and sympathetic chain.

### DESCRIPTION OF A THIRD EMBODIMENT OF AN INTERVERTEBRAL PROSTHETIC DISC

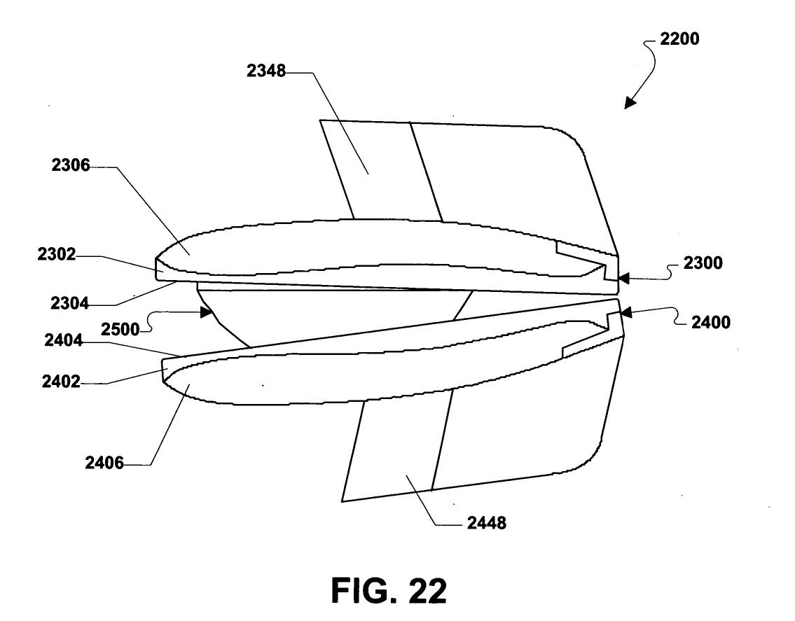

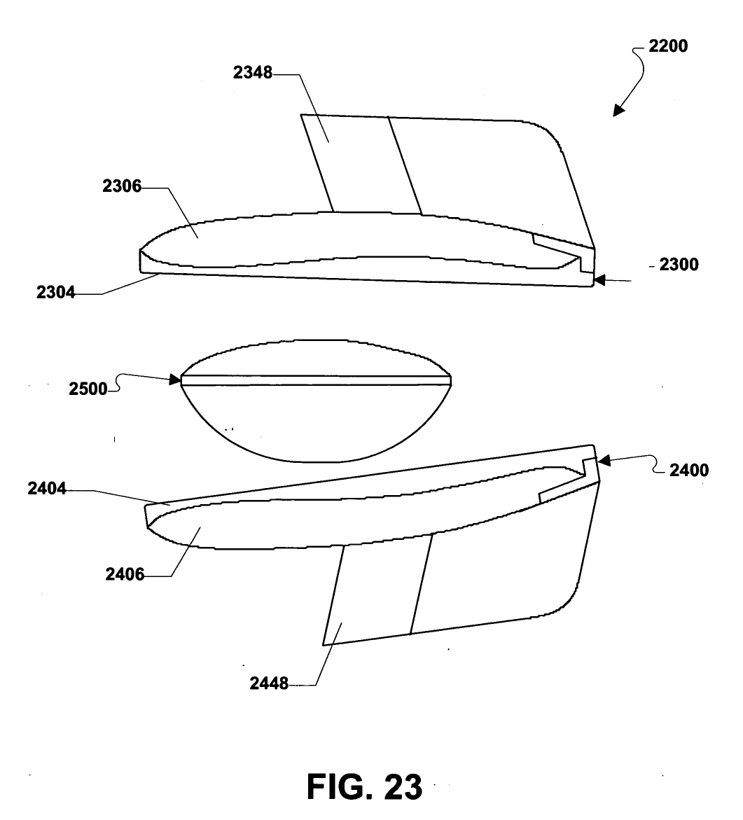

Referring to FIGS. 22 through 26 a third embodiment of an intervertebral prosthetic disc is shown and is generally designated 2200. As illustrated, the intervertebral prosthetic disc 2200 can include a superior component 2300, an inferior component 2400, and a nucleus 2500 disposed, or otherwise installed, there between. In a particular embodiment, the components 2300, 2400 and the nucleus 2500 can be made from one or more biocompatible materials. For example, the materials can be metal containing materials, polymer materials, or composite materials that include metals, polymers, or combinations of metals and polymers. Additionally, the biocompatible materials can include, or contain, an inorganic carbon-based material, such as graphite.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 2300, 2400 can be made from any other substantially rigid biocompatible materials.

In a particular embodiment, the superior component 2300 can include a superior support plate 2302 that has a superior articular surface 2304 and a superior bearing surface 2306. In a particular embodiment, the superior articular surface 2304 can be substantially flat and the superior bearing surface 2306 can be generally curved. In an alternative embodiment, at least a portion of the superior articular surface 2304 can be generally curved and the superior bearing surface 2306 can be substantially flat.

In a particular embodiment, after installation, the superior bearing surface 2306 can be in direct contact with vertebral bone, e.g., cortical bone and cancellous bone. Further, the superior bearing surface 2306 can be coated with a bone-growth promoting substance, e.g., a hydroxyapatite coating formed of calcium phosphate. Additionally, the superior bearing surface 2306 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

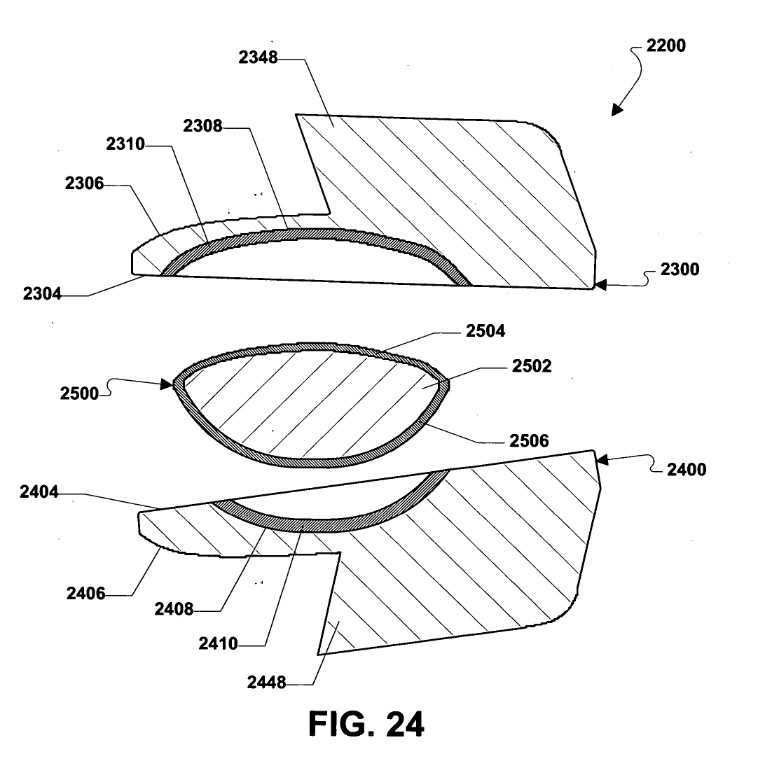

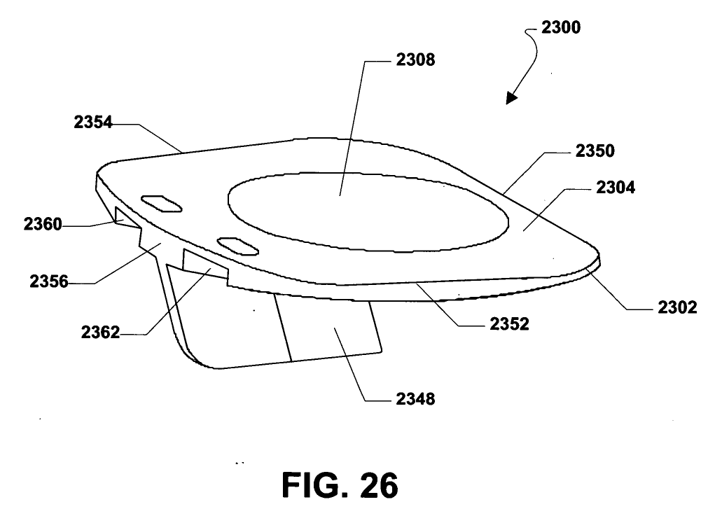

As illustrated in FIG. 24 and FIG. 26, a superior depression 2308 is established within the superior articular surface 2304 of the superior support plate 2302. In a particular embodiment, the superior depression 2308 has an arcuate shape. For example, the superior depression 2308 can have a hemispherical shape, an elliptical shape, a cylindrical shape, or any combination thereof.

FIG. 24 shows that a superior wear resistant layer 2310 can be disposed within, or deposited within, the superior depression 2308. In a particular embodiment, the superior wear resistant layer 2310 is substantially wear resistant. Further, in a particular embodiment, the superior wear resistant layer 2310 can include pyrolytic carbon.

FIG. 22 through FIG. 26 indicate that the superior component 2300 can include a superior keel 2348 that extends from superior bearing surface 2306. During installation, described below, the superior keel 2348 can at least partially engage a keel groove that can be established within a cortical rim of a superior vertebra. Further, the superior keel 2348 can be coated with a bone-growth promoting substance, e.g., a hydroxyapatite coating formed of calcium phosphate. In a particular embodiment, the superior keel 2348 does not include proteins, e.g., bone morphogenetic protein (BMP). Additionally, the superior keel 2348 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the superior component 2300, depicted in FIG. 26, can be generally rectangular in shape. For example, the superior component 2300 can have a substantially straight posterior side 2350. A first substantially straight lateral side 2352 and a second substantially straight lateral side 2354 can extend substantially perpendicularly from the posterior side 2350 to an anterior side 2356. In a particular embodiment, the anterior side 2356 can curve outward such that the superior component 2300 is wider through the middle than along the lateral sides 2352, 2354. Further, in a particular embodiment, the lateral sides 2352, 2354 are substantially the same length.

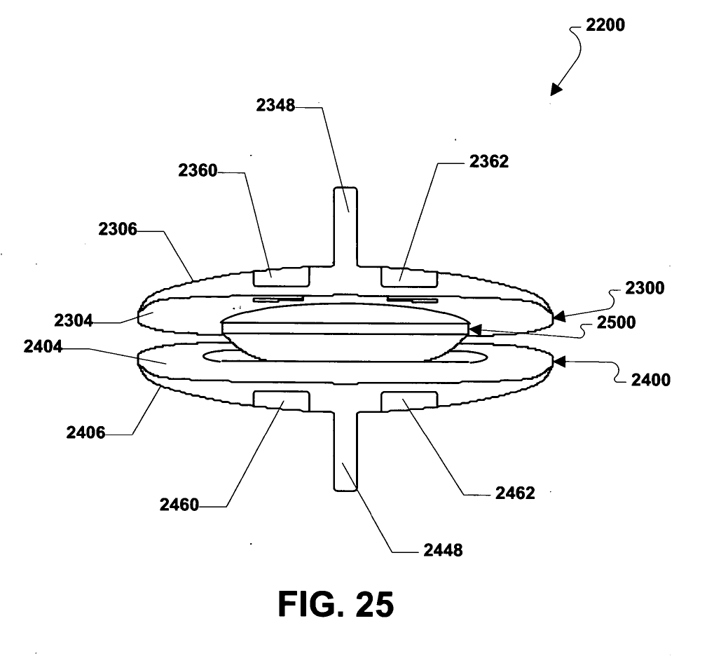

FIG. 25 shows that the superior component 2300 can include a first implant inserter engagement hole 2360 and a second implant inserter engagement hole 2362. In a particular embodiment, the implant inserter engagement holes 2360, 2362 are configured to receive a correspondingly shaped arm that extends from an implant inserter (not shown) that can be used to facilitate the proper installation of an intervertebral prosthetic disc, e.g., the intervertebral prosthetic disc 2200 shown in FIG. 22 through FIG. 27.

In a particular embodiment, the inferior component 2400 can include an inferior support plate 2402 that has an inferior articular surface 2404 and an inferior bearing surface 2406. In a particular embodiment, the inferior articular surface 2404 can be substantially flat and the inferior bearing surface 2406 can be generally curved. In an alternative embodiment, at least a portion of the inferior articular surface 2404 can be generally curved and the inferior bearing surface 2406 can be substantially flat.

In a particular embodiment, after installation, the inferior bearing surface 2406 can be in direct contact with vertebral bone, e.g., cortical bone and cancellous bone. Further, the inferior bearing surface 2406 can be coated with a bone-growth promoting substance, e.g., a hydroxyapatite coating formed of calcium phosphate. Additionally, the inferior bearing surface 2406 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

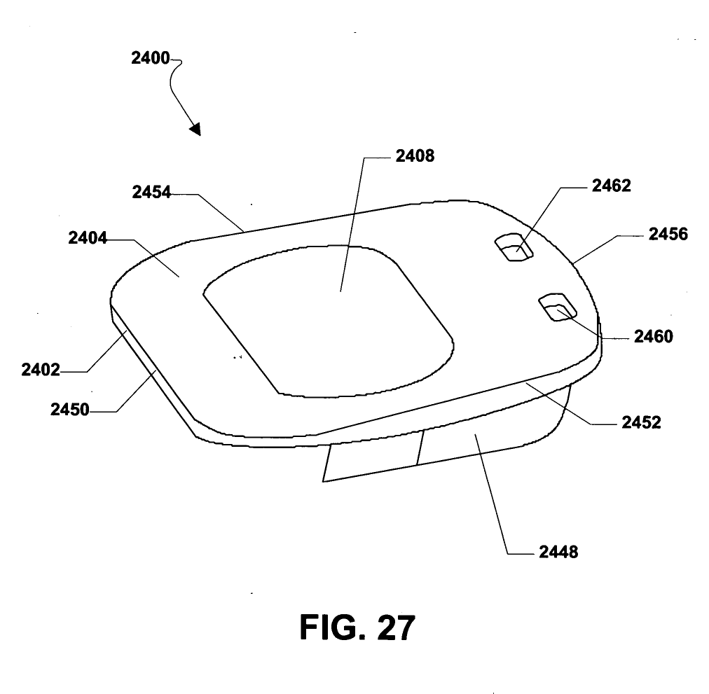

As illustrated in FIG. 24 and FIG. 27, an inferior depression 2408 is established within the inferior articular surface 2404 of the inferior support plate 2402. In a particular embodiment, the inferior depression 2408 has an arcuate shape. For example, the inferior depression 2408 can have a hemispherical shape, an elliptical shape, a cylindrical shape, or any combination thereof.

FIG. 24 shows that an inferior wear resistant layer 2410 can be disposed within, or deposited within, the inferior depression 2408. In a particular embodiment, the inferior wear resistant layer 2410 is substantially wear resistant. Further, in a particular embodiment, the inferior wear resistant layer 2410 can include pyrolytic carbon.

FIG. 22 through FIG. 25 and FIG. 27 indicate that the inferior component 2400 can include an inferior keel 2448 that extends from inferior bearing surface 2406. During installation, described below, the inferior keel 2448 can at least partially engage a keel groove that can be established within a cortical rim of a vertebra. Further, the inferior keel 2448 can be coated with a bone-growth promoting substance, e.g., a hydroxyapatite coating formed of calcium phosphate. In a particular embodiment, the inferior keel 2448 does not include proteins, e.g., bone morphogenetic protein (BMP). Additionally, the inferior keel 2448 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the inferior component 2400, shown in FIG. 27, can be shaped to match the shape of the superior component 2300, shown in FIG. 26. Further, the inferior component 2400 can be generally rectangular in shape. For example, the inferior component 2400 can have a substantially straight posterior side 2450. A first substantially straight lateral side 2452 and a second substantially straight lateral side 2454 can extend substantially perpendicularly from the posterior side 2450 to an anterior side 2456. In a particular embodiment, the anterior side 2456 can curve outward such that the inferior component 2400 is wider through the middle than along the lateral sides 2452, 2454. Further, in a particular embodiment, the lateral sides 2452, 2454 are substantially the same length.

FIG. 25 shows that the inferior component 2400 can include a first implant inserter engagement hole 2460 and a second implant inserter engagement hole 2462. In a particular embodiment, the implant inserter engagement holes 2460, 2462 are configured to receive a correspondingly shaped arm that extends from an implant inserter (not shown) that can be used to facilitate the proper installation of an intervertebral prosthetic disc, e.g., the intervertebral prosthetic disc 2200 shown in FIG. 22 through FIG. 27.

FIG. 24 shows that the nucleus 2500 can include a core 2502. A superior wear resistant layer 2504 can be deposited on, or affixed to, the core 2502. Also, an inferior resistant layer 2506 can be deposited on, or affixed to, the core 2502. In a particular embodiment, the core 2502 can include an inorganic carbon-based material, such as graphite. Further, in a particular embodiment, the superior wear resistant layer 2504 and the inferior wear resistant layer 2506 can include pyrolytic carbon. Additionally, the superior wear resistant layer 2504 and the inferior wear resistant layer 2506 can each have an arcuate shape. For example, the superior wear resistant layer 2504 of the nucleus 2500 and the inferior wear resistant layer 2506 of the nucleus 2500 can have a hemispherical shape, an elliptical shape, a cylindrical shape, or any combination thereof. Further, in a particular embodiment, the superior wear resistant layer 2504 can be curved to match the superior depression 2308 of the superior component 2300. Also, in a particular embodiment, the inferior wear resistant layer 2506 of the nucleus 2500 can be curved to match the inferior depression 2408 of the inferior component 2400.

As shown in FIG. 22, the superior wear resistant layer 2504 of the nucleus 2500 can engage the superior wear resistant layer 2310 within the superior depression 2308 and can allow relative motion between the superior component 2300 and the nucleus 2500. Also, the inferior wear resistant layer 2506 of the nucleus 2500 can engage the inferior wear resistant layer 2410 within the inferior depression 2408 and can allow relative motion between the inferior component 2400 and the nucleus 2500. Accordingly, the nucleus 2500 can engage the superior component 2300 and the inferior component 2400 and the nucleus 2500 can allow the superior component 2300 to rotate with respect to the inferior component 2400.

In a particular embodiment, the overall height of the intervertebral prosthetic device 2200 can be in a range from fourteen millimeters to forty-six millimeters (14-46 mm). Further, the installed height of the intervertebral prosthetic device 2200 can be in a range from eight millimeters to sixteen millimeters (8-16 mm). In a particular embodiment, the installed height can be substantially equivalent to the distance between an inferior vertebra and a superior vertebra when the intervertebral prosthetic device 2200 is installed there between.

In a particular embodiment, the length of the intervertebral prosthetic device 2200, e.g., along a longitudinal axis, can be in a range from thirty millimeters to forty millimeters (30-40 mm). Additionally, the width of the intervertebral prosthetic device 2200, e.g., along a lateral axis, can be in a range from twenty-five millimeters to forty millimeters (25-40 mm).

### DESCRIPTION OF A FOURTH EMBODIMENT OF AN INTERVERTEBRAL PROSTHETIC DISC

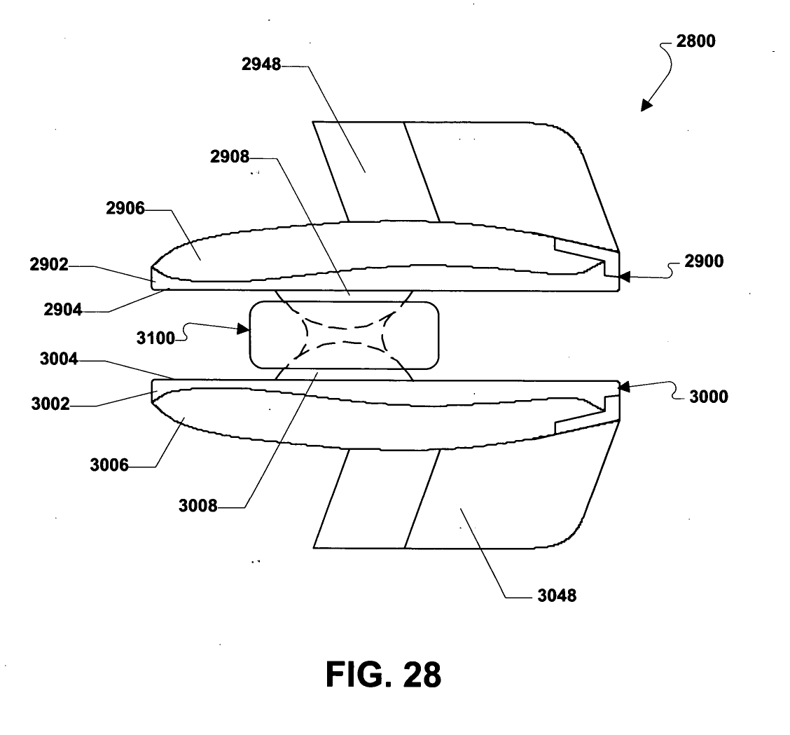

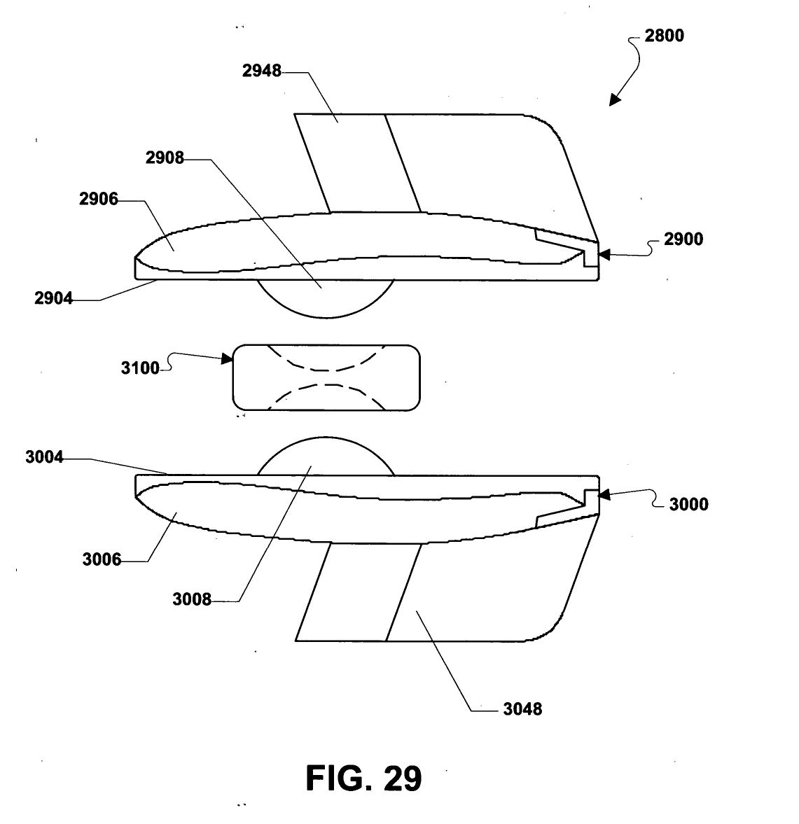

Referring to FIGS. 28 through 33, a fourth embodiment of an intervertebral prosthetic disc is shown and is generally designated 2800. As illustrated, the intervertebral prosthetic disc 2800 can include a superior component 2900, an inferior component 3000, and a nucleus 3100 disposed, or otherwise installed, there between. In a particular embodiment, the components 2900, 3000 and the nucleus 3100 can be made from one or more biocompatible materials. For example, the materials can be metal containing materials, polymer materials, or composite materials that include metals, polymers, or combinations of metals and polymers. Additionally, the biocompatible materials can include, or contain, an inorganic carbon-based material, such as graphite.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 2900, 3000 can be made from any other substantially rigid biocompatible materials.

In a particular embodiment, the superior component 2900 can include a superior support plate 2902 that has a superior articular surface 2904 and a superior bearing surface 2906. In a particular embodiment, the superior articular surface 2904 can be substantially flat and the superior bearing surface 2906 can be generally curved. In an alternative embodiment, at least a portion of the superior articular surface 2904 can be generally curved and the superior bearing surface 2906 can be substantially flat.

In a particular embodiment, after installation, the superior bearing surface 2906 can be in direct contact with vertebral bone, e.g., cortical bone and cancellous bone. Further, the superior bearing surface 2906 can be coated with a bone-growth promoting substance, e.g., a hydroxyapatite coating formed of calcium phosphate. Additionally, the superior bearing surface 2906 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 28 through FIG. 32, a superior projection 2908 extends from the superior articular surface 2904 of the superior support plate 2902. In a particular embodiment, the superior projection 2908 has an arcuate shape. For example, the superior depression 2908 can have a hemispherical shape, an elliptical shape, a cylindrical shape, or any combination thereof.

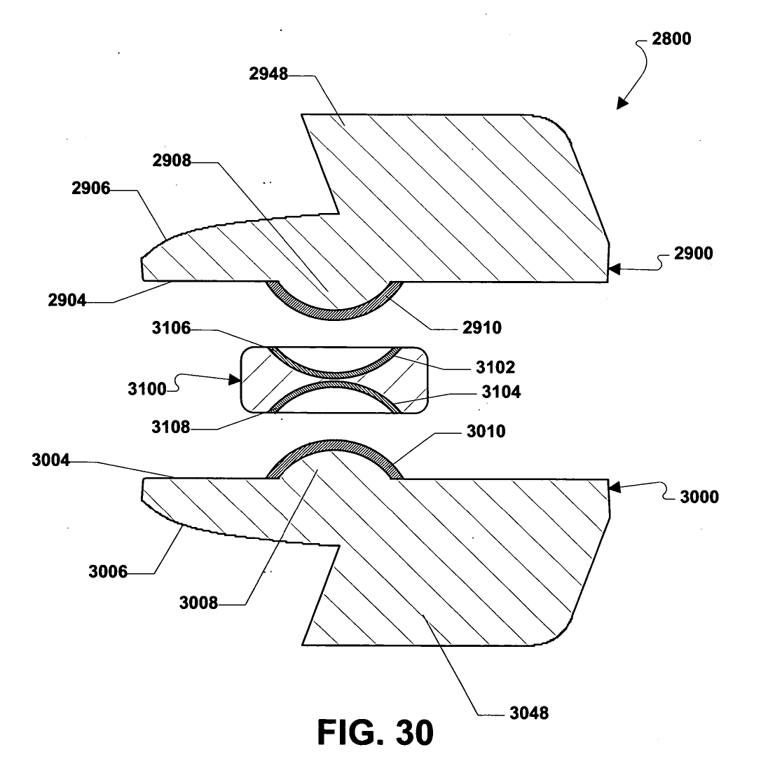

FIG. 30 shows that the superior projection 2908 can include a superior wear resistant layer 2910. In a particular embodiment, the superior wear resistant layer 2910 can be attached to, affixed to, or otherwise deposited on, the superior projection 2908. In a particular embodiment, the superior wear resistant layer 2910 is substantially wear resistant. Further, in a particular embodiment, the superior wear resistant layer 2910 can be pyrolytic carbon.

FIG. 28 through FIG. 32 indicate that the superior component 2900 can include a superior keel 2948 that extends from superior bearing surface 2906. During installation, described below, the superior keel 2948 can at least partially engage a keel groove that can be established within a cortical rim of a superior vertebra. Further, the superior keel 2948 can be coated with a bone-growth promoting substance, e.g., a hydroxyapatite coating formed of calcium phosphate. In a particular embodiment, the superior keel 2948 does not include proteins, e.g., bone morphogenetic protein (BMP). Additionally, the superior keel 2948 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

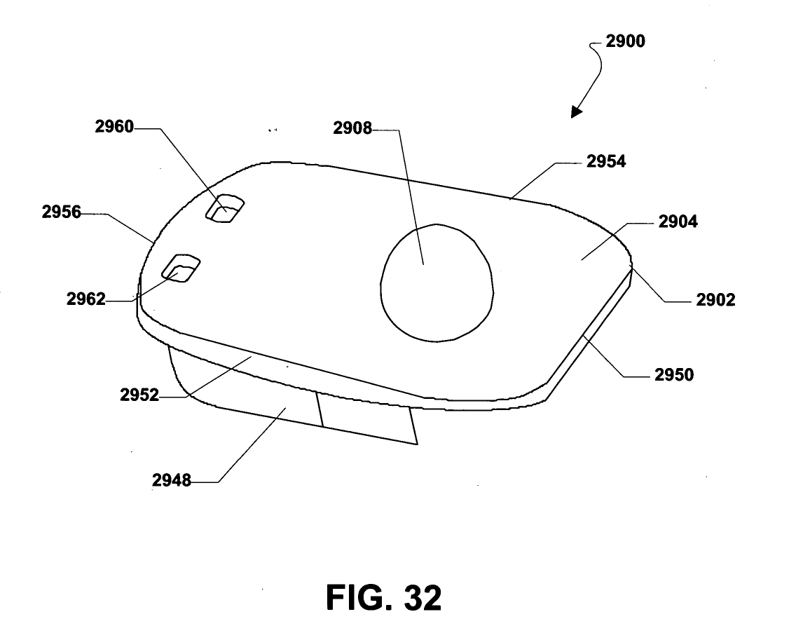

In a particular embodiment, the superior component 2900, depicted in FIG. 32, can be generally rectangular in shape. For example, the superior component 2900 can have a substantially straight posterior side 2950. A first substantially straight lateral side 2952 and a second substantially straight lateral side 2954 can extend substantially perpendicularly from the posterior side 2950 to an anterior side 2956. In a particular embodiment, the anterior side 2956 can curve outward such that the superior component 2900 is wider through the middle than along the lateral sides 2952, 2954. Further, in a particular embodiment, the lateral sides 2952, 2954 are substantially the same length.

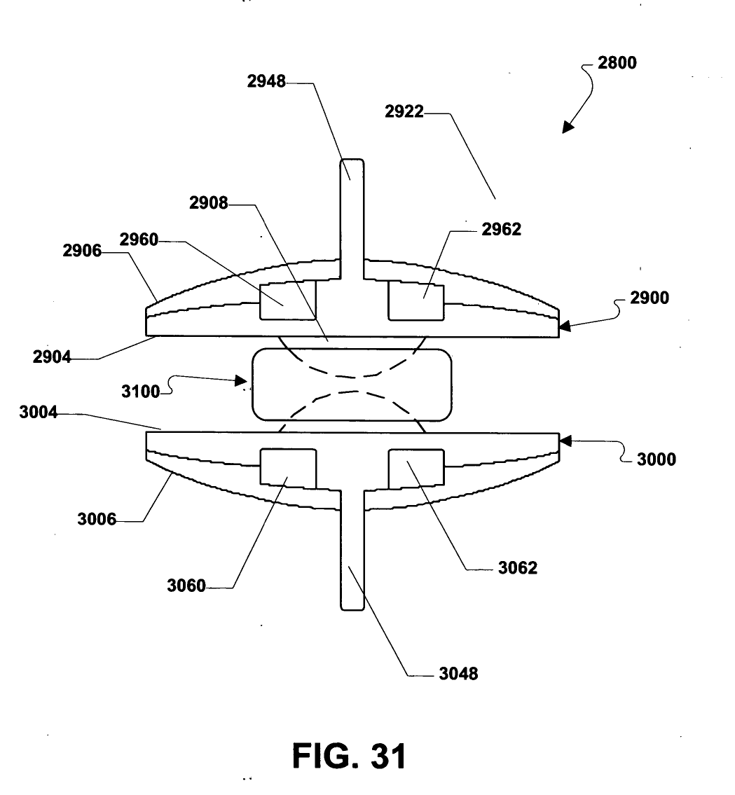

FIG. 31 shows that the superior component 2900 can include a first implant inserter engagement hole 2960 and a second implant inserter engagement hole 2962. In a particular embodiment, the implant inserter engagement holes 2960, 2962 are configured to receive a correspondingly shaped arm that extends from an implant inserter (not shown) that can be used to facilitate the proper installation of an intervertebral prosthetic disc, e.g., the intervertebral prosthetic disc 2200 shown in FIG. 28 through FIG. 33.

In a particular embodiment, the inferior component 3000 can include an inferior support plate 3002 that has an inferior articular surface 3004 and an inferior bearing surface 3006. In a particular embodiment, the inferior articular surface 3004 can be substantially flat and the inferior bearing surface 3006 can be generally curved. In an alternative embodiment, at least a portion of the inferior articular surface 3004 can be generally curved and the inferior bearing surface 3006 can be substantially flat.

In a particular embodiment, after installation, the inferior bearing surface 3006 can be in direct contact with vertebral bone, e.g., cortical bone and cancellous bone. Further, the inferior bearing surface 3006 can be coated with a bone-growth promoting substance, e.g., a hydroxyapatite coating formed of calcium phosphate. Additionally, the inferior bearing surface 3006 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 28 through FIG. 31 and FIG. 33, an inferior projection 3008 can extend from the inferior articular surface 3004 of the inferior support plate 3002. In a particular embodiment, the inferior projection 3008 has an arcuate shape. For example, the inferior projection 3008 can have a hemispherical shape, an elliptical shape, a cylindrical shape, or any combination thereof.

FIG. 30 shows that the inferior projection 3008 can include an inferior wear resistant layer 3010. In a particular embodiment, the inferior wear resistant layer 3010 can be attached to, affixed to, or otherwise deposited on, the inferior projection 3008. In a particular embodiment, the inferior wear resistant layer 3010 is substantially wear resistant. Further, in a particular embodiment, the inferior wear resistant layer 3010 can be pyrolytic carbon.

FIG. 28 through FIG. 31 and FIG. 33 indicate that the inferior component 3000 can include an inferior keel 3048 that extends from inferior bearing surface 3006. During installation, described below, the inferior keel 3048 can at least partially engage a keel groove that can be established within a cortical rim of a vertebra. Further, the inferior keel 3048 can be coated with a bone-growth promoting substance, e.g., a hydroxyapatite coating formed of calcium phosphate. In a particular embodiment, the inferior keel 3048 does not include proteins, e.g., bone morphogenetic protein (BMP). Additionally, the inferior keel 3048 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

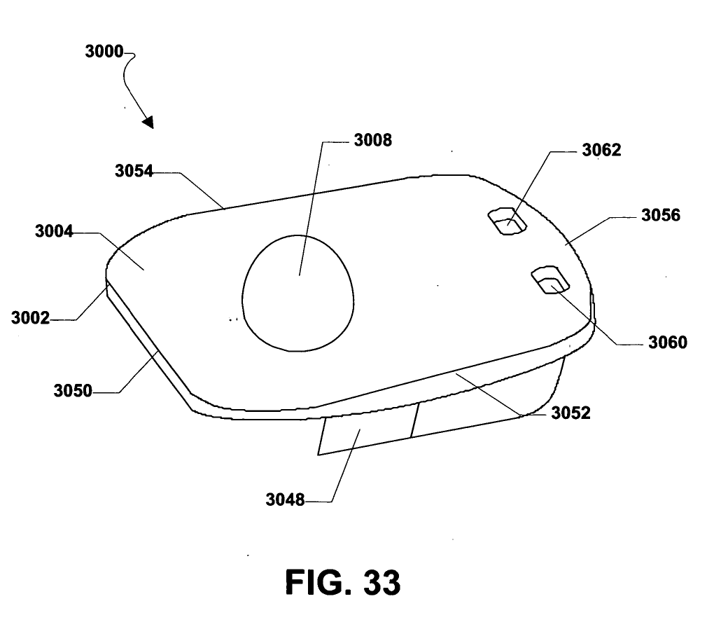

In a particular embodiment, the inferior component 3000, shown in FIG. 33, can be shaped to match the shape of the superior component 2900, shown in FIG. 32. Further, the inferior component 3000 can be generally rectangular in shape. For example, the inferior component 3000 can have a substantially straight posterior side 3050. A first substantially straight lateral side 3052 and a second substantially straight lateral side 3054 can extend substantially perpendicularly from the posterior side 3050 to an anterior side 3056. In a particular embodiment, the anterior side 3056 can curve outward such that the inferior component 3000 is wider through the middle than along the lateral sides 3052, 3054. Further, in a particular embodiment, the lateral sides 3052, 3054 are substantially the same length.

FIG. 31 shows that the inferior component 3000 can include a first implant inserter engagement hole 3060 and a second implant inserter engagement hole 3062. In a particular embodiment, the implant inserter engagement holes 3060, 3062 are configured to receive a correspondingly shaped arm that extends from an implant inserter (not shown) that can be used to facilitate the proper installation of an intervertebral prosthetic disc, e.g., the intervertebral prosthetic disc 2200 shown in FIG. 28 through FIG. 33.

FIG. 30 shows that the nucleus 3100 can include a superior depression 3102 and an inferior depression 3104. In a particular embodiment, the superior depression 3102 and the inferior depression 3104 can each have an arcuate shape. For example, the superior depression 3102 of the nucleus 3100 and the inferior depression 3104 of the nucleus 3100 can have a hemispherical shape, an elliptical shape, a cylindrical shape, or any combination thereof. Further, in a particular embodiment, the superior depression 3102 can be curved to match the superior projection 2908 of the superior component 2900. Also, in a particular embodiment, the inferior depression 3104 of the nucleus 3100 can be curved to match the inferior projection 3008 of the inferior component 3000.

FIG. 30 shows that a superior wear resistant layer 3106 can be disposed within, or deposited within, the superior depression 3102 of the nucleus 3100. Also, an inferior wear resistant layer 3108 can be disposed within, or deposited within, the inferior depression 3103 of the nucleus 3100. In a particular embodiment, the superior wear resistant layer 3106 and the inferior wear resistant layer 3108 is substantially wear resistant. Further, in a particular embodiment, the superior wear resistant layer 3106 and the inferior wear resistant layer 3108 can be pyrolytic carbon.

As shown in FIG. 28, the superior wear resistant layer 3106 of the nucleus 3100 can engage the superior wear resistant layer 2910 of the superior component 2900 and can allow relative motion between the superior component 2900 and the nucleus 3100. Also, the inferior wear resistant layer 3108 of the nucleus 3100 can engage the inferior wear resistant layer 3010 of the inferior component 3000 and can allow relative motion between the inferior component 3000 and the nucleus 3100. Accordingly, the nucleus 3100 can engage the superior component 2900 and the inferior component 3000, and the nucleus 3100 can allow the superior component 2900 to rotate with respect to the inferior component 3000.

In a particular embodiment, the overall height of the intervertebral prosthetic device 2800 can be in a range from fourteen millimeters to forty-six millimeters (14-46 mm). Further, the installed height of the intervertebral prosthetic device 2800 can be in a range from eight millimeters to sixteen millimeters (8-16 mm). In a particular embodiment, the installed height can be substantially equivalent to the distance between an inferior vertebra and a superior vertebra when the intervertebral prosthetic device 2800 is installed there between.

In a particular embodiment, the length of the intervertebral prosthetic device 2800, e.g., along a longitudinal axis, can be in a range from thirty millimeters to forty millimeters (30-40 mm). Additionally, the width of the intervertebral prosthetic device 2800, e.g., along a lateral axis, can be in a range from twenty-five millimeters to forty millimeters (25-40 mm).

### DESCRIPTION OF A FIFTH EMBODIMENT OF AN INTERVERTEBRAL PROSTHETIC DISC

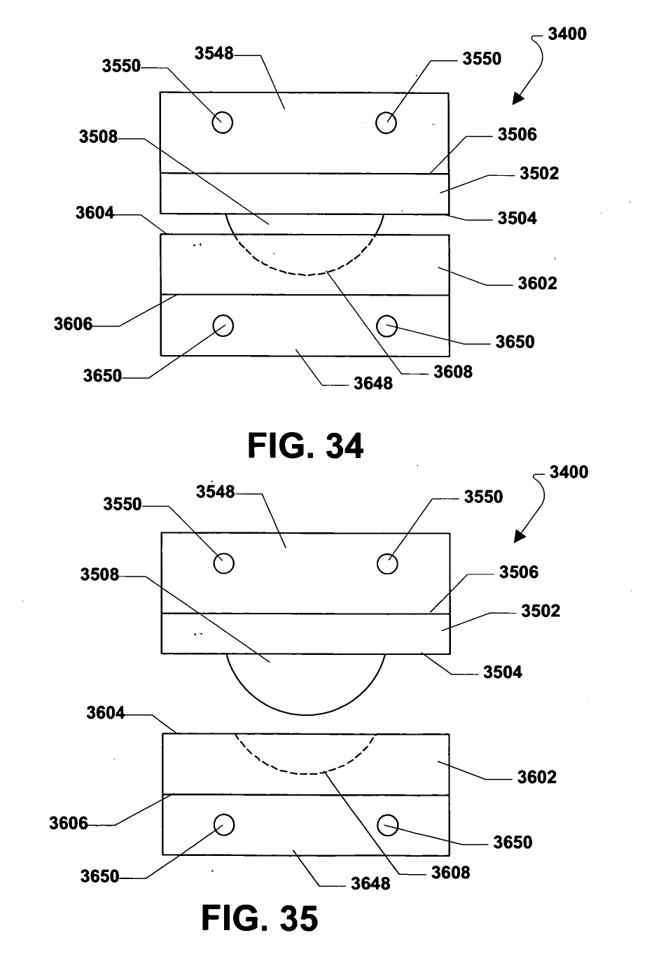

Referring to FIGS. 34 through 38 a fifth embodiment of an intervertebral prosthetic disc is shown and is generally designated 3400. As illustrated, the intervertebral prosthetic disc 3400 can include a superior component 3500 and an inferior component 3600. In a particular embodiment, the components 3500, 3600 can be made from one or more biocompatible materials. For example, the materials can be metal containing materials, polymer materials, or composite materials that include metals, polymers, or combinations of metals and polymers. Additionally, the biocompatible materials can include, or contain, an inorganic carbon-based material, such as graphite.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile, (PAN), polyvinylacrylate (PVA), polyvinylpyrrolidone (PVP), or a combination thereof. Alternatively, the components 3500, 3600 can be made from any other substantially rigid biocompatible materials.

In a particular embodiment, the superior component 3500 can include a superior support plate 3502 that has a superior articular surface 3504 and a superior bearing surface 3506. In a particular embodiment, the superior articular surface 3504 can be substantially flat and the superior bearing surface 3506 can be substantially flat. In an alternative embodiment, at least a portion of the superior articular surface 3504 can be generally curved and at least a portion of the superior bearing surface 3506 can be generally curved.

As illustrated in FIG. 34 through FIG. 36, a projection 3508 extends from the superior articular surface 3504 of the superior support plate 3502. In a particular embodiment, the projection 3508 has a hemi-spherical shape. Alternatively, the projection 3508 can have an elliptical shape, a cylindrical shape, or other arcuate shape.

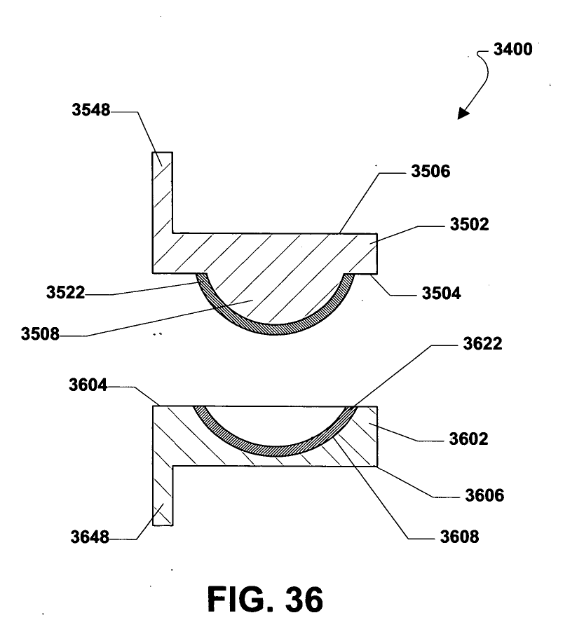

Referring to FIG. 36, the projection 3508 can include a superior wear resistant layer 3522 affixed to, deposited on, or otherwise disposed thereon. In a particular embodiment, the superior wear resistant layer 3522 can be pyrolytic carbon.

FIG. 34 through FIG. 36 also show that the superior component 3500 can include a superior bracket 3548 that can extend substantially perpendicular from the superior support plate 4502. Further, the superior bracket 3548 can include at least one hole 3550. In a particular embodiment, a fastener, e.g., a screw, can be inserted through the hole 3550 in the superior bracket 4548 in order to attach, or otherwise affix, the superior component 4500 to a superior vertebra.

The superior bearing surface 3506 can be coated with a bone-growth promoting substance, e.g., a hydroxyapatite coating formed of calcium phosphate. Additionally, the superior bearing surface 3506 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

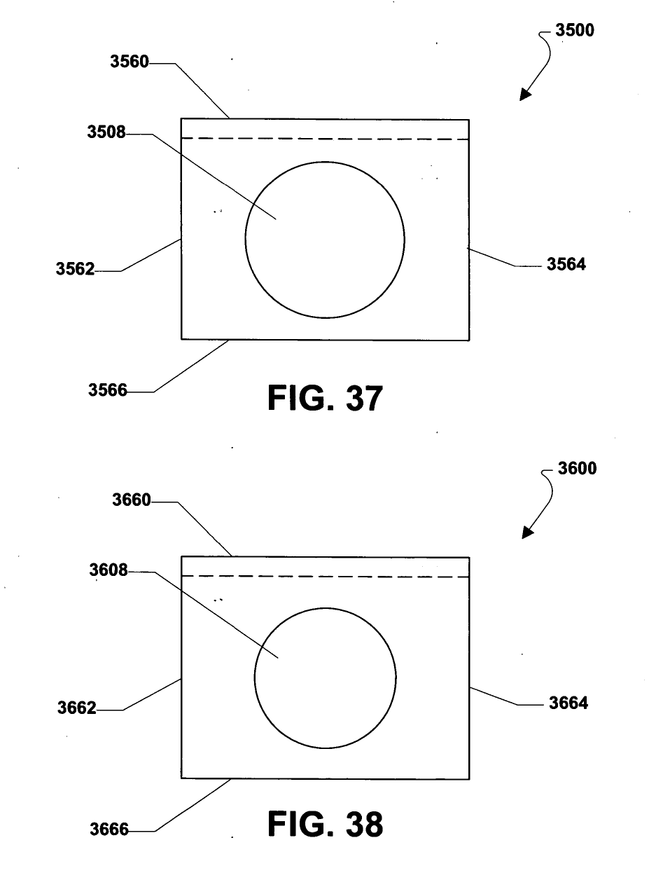

As illustrated in FIG. 37, the superior component 3500 can be generally rectangular in shape. For example, the superior component 3500 can have a substantially straight posterior side 3560. A first straight lateral side 3562 and a second substantially straight lateral side 3564 can extend substantially perpendicular from the posterior side 3560 to a substantially straight anterior side 3566. In a particular embodiment, the anterior side 3566 and the posterior side 3560 are substantially the same length. Further, in a particular embodiment, the lateral sides 3562, 3564 are substantially the same length.

In a particular embodiment, the inferior component 3600 can include an inferior support plate 3602 that has an inferior articular surface 3604 and an inferior bearing surface 3606. In a particular embodiment, the inferior articular surface 3604 can be generally curved and the inferior bearing surface 3606 can be substantially flat. In an alternative embodiment, the inferior articular surface 3604 can be substantially flat and at least a portion of the inferior bearing surface 3606 can be generally curved.

As illustrated in FIG. 34 through FIG. 36, a depression 3608 extends into the inferior articular surface 3604 of the inferior support plate 3602. In a particular embodiment, the depression 3608 is sized and shaped to receive the projection 3508 of the superior component 3500. For example, the depression 3608 can have a hemi-spherical shape. Alternatively, the depression 3608 can have an elliptical shape, a cylindrical shape, or other arcuate shape.

Referring to FIG. 36, the depression 3608 can include a substantially inferior wear resistant layer 3622 that is deposited, or disposed, within the depression 3608. In a particular embodiment, the inferior wear resistant layer 3622 can be pyrolytic carbon.

FIG. 34 through FIG. 36 also show that the inferior component 3600 can include an inferior bracket 3648 that can extend substantially perpendicular from the inferior support plate 4502. Further, the inferior bracket 3648 can include a hole 3650. In a particular embodiment, a fastener, e.g., a screw, can be inserted through the hole 3650 in the inferior bracket 4548 in order to attach, or otherwise affix, the inferior component 4500 to an inferior vertebra.

The inferior bearing surface 3606 can be coated with a bone-growth promoting substance, e.g., a hydroxyapatite coating formed of calcium phosphate. Additionally, the inferior bearing surface 3606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 38, the inferior component 3600 can be generally rectangular in shape. For example, the inferior component 3600 can have a substantially straight posterior side 3660. A first straight lateral side 3662 and a second substantially straight lateral side 3664 can extend substantially perpendicular from the posterior side 3660 to a substantially straight anterior side 3666. In a particular embodiment, the anterior side 3666 and the posterior side 3660 are substantially the same length. Further, in a particular embodiment, the lateral sides 3662, 3664 are substantially the same length.

In a particular embodiment, the overall height of the intervertebral prosthetic device 3400 can be in a range from fourteen millimeters to forty-six millimeters (14-46 mm). Further, the installed height of the intervertebral prosthetic device 3400 can be in a range from eight millimeters to sixteen millimeters (8-16 mm). In a particular embodiment, the installed height can be substantially equivalent to the distance between an inferior vertebra and a superior vertebra when the intervertebral prosthetic device 3400 is installed there between.

In a particular embodiment, the length of the intervertebral prosthetic device 3400, e.g., along a longitudinal axis, can be in a range from thirty millimeters to forty millimeters (30-40 mm). Additionally, the width of the intervertebral prosthetic device 3400, e.g., along a lateral axis, can be in a range from twenty-five millimeters to forty millimeters (25-40 mm). Moreover, in a particular embodiment, each bracket 3548, 3648 can have a height in a range from three millimeters to fifteen millimeters (3-15 mm).

### DESCRIPTION OF A SIXTH EMBODIMENT OF AN INTERVERTEBRAL PROSTHETIC DISC

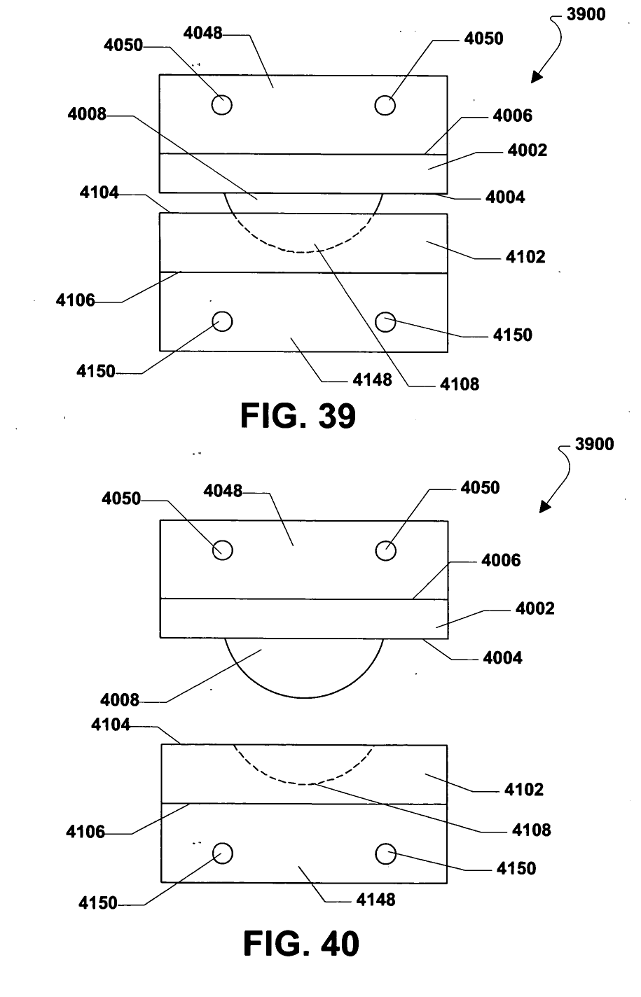

Referring to FIGS. 39 through 43 a sixth embodiment of an intervertebral prosthetic disc is shown and is generally designated 3900. As illustrated, the intervertebral prosthetic disc 3900 can include a superior component 4000 and an inferior component 4100. In a particular embodiment, the components 4000, 4100 can be made from one or more biocompatible materials. For example, the materials can be metal containing materials, polymer materials, or composite materials that include metals, polymers, or combinations of metals and polymers. Additionally, the biocompatible materials can include, or contain, an inorganic carbon-based material, such as graphite.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 4000, 4100 can be made from any other substantially rigid biocompatible materials.

In a particular embodiment, the superior component 4000 can include a superior support plate 4002 that has a superior articular surface 4004 and a superior bearing surface 4006. In a particular embodiment, the superior articular surface 4004 can be substantially flat and the superior bearing surface 4006 can be substantially flat. In an alternative embodiment, at least a portion of the superior articular surface 4004 can be generally curved and at least a portion of the superior bearing surface 4006 can be generally curved.

As illustrated in FIG. 39 through FIG. 41, a projection 4008 extends from the superior articular surface 4004 of the superior support plate 4002. In a particular embodiment, the projection 4008 has a hemi-spherical shape. Alternatively, the projection 4008 can have an elliptical shape, a cylindrical shape, or other arcuate shape.

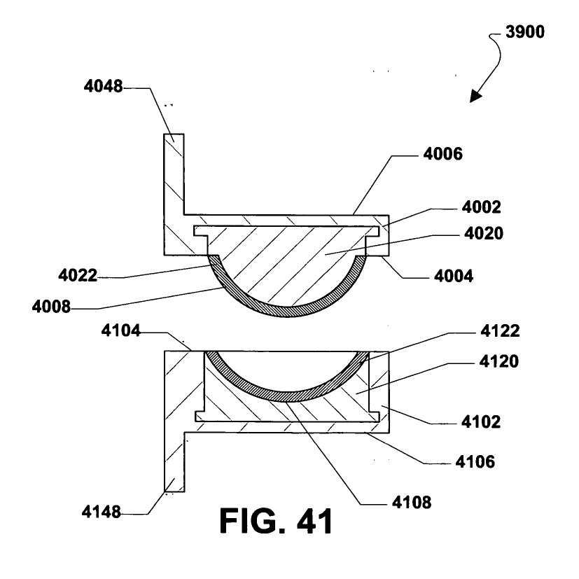

Referring to FIG. 41, the projection 4008 can include a base 4020 and a superior wear resistant layer 4022 affixed to, deposited on, or otherwise disposed on, the base 4020. In a particular embodiment, the base 4020 can act as a substrate and the superior wear resistant layer 4022 can be deposited on the base 4020. Further, the base 4020 can engage a cavity 4024 that can be formed in the superior support plate 4002. In a particular embodiment, the cavity 4024 can be sized and shaped to receive the base 4020 of the projection 4008. Further, the base 4020 of the projection 4008 can be press fit into the cavity 4024.

In a particular embodiment, the base 4020 of the projection can be made from graphite. Further, in a particular embodiment, the superior wear resistant layer 4022 can be pyrolytic carbon that is deposited on the base 4020. As such, the base 4020 can be made from a material that can allow pyrolytic carbon to be deposited thereon. Thereafter, the base 4020 can be fitted into a superior support plate 4002 made from one or more of the materials described herein. Accordingly, the superior support plate 4002 may be made from a material that does not facilitate the deposition of pyrolytic carbon thereon.

Also, in a particular embodiment, the base 4020 can be roughened prior to the deposition of the pyrolytic carbon thereon. For example, the base 4020 can be roughened using a roughening process. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 4020 on which the pyrolytic carbon is deposited can be serrated and can include one or more teeth, spikes, or other protrusions extending therefrom. The serrations of the base 4020 can facilitate anchoring of the pyrolytic carbon on the base 4020 and can substantially reduce the likelihood of delamination of the superior wear resistant layer 4022 from the base 4020.

In a particular embodiment, the superior wear resistant layer 4022 can have a thickness in a range of fifty micrometers to five millimeters (50 μm-5 mm). Further, the superior wear resistant layer 4022 can have a thickness in a range of two hundred micrometers to two millimeters (200 μm-2 mm). In a particular embodiment, the serrations that can be formed on the surface of the base 4020 can have a height that is at most half of the thickness of the superior wear resistant layer 4022. Accordingly, the likelihood that the serrations will protrude through the superior wear resistant layer 4022 is substantially minimized.

Additionally, in a particular embodiment, a Young's modulus of the superior wear resistant layer 4022 can be substantially greater than a Young's modulus of the base 4020. Also, a hardness of the superior wear resistant layer 4022 can be substantially greater than a hardness of the base 4020. Further, a toughness of the superior wear resistant layer 4022 can be substantially greater than a toughness of the base 4020. In a particular embodiment, the superior wear resistant layer 4022 can be annealed immediately after deposition in order to minimize cracking of the superior wear resistant layer. Also, the superior wear resistant layer 4022 can be polished in order to minimize surface irregularities of the superior wear resistant layer 4022 and increase a smoothness of the superior wear resistant layer 4022.

FIG. 39 through FIG. 41 also show that the superior component 4000 can include a superior bracket 4048 that can extend substantially perpendicular from the superior support plate 4502. Further, the superior bracket 4048 can include a hole 4050. In a particular embodiment, a fastener, e.g., a screw, can be inserted through the hole 4050 in the superior bracket 4548 in order to attach, or otherwise affix, the superior component 4500 to a superior vertebra.

The superior bearing surface 4006 can be coated with a bone-growth promoting substance, e.g., a hydroxyapatite coating formed of calcium phosphate. Additionally, the superior bearing surface 4006 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

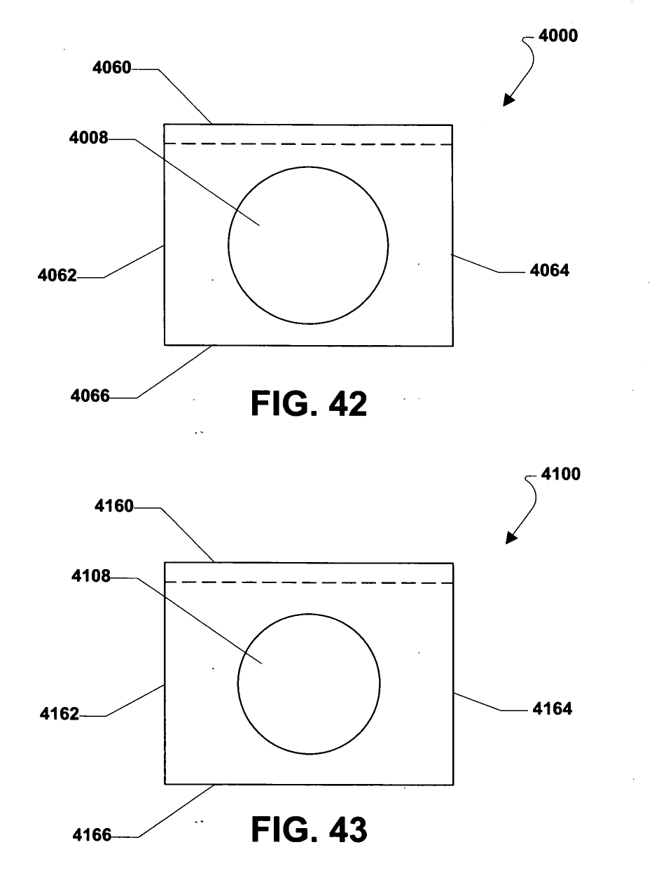

As illustrated in FIG. 42, the superior component 4000 can be generally rectangular in shape. For example, the superior component 4000 can have a substantially straight posterior side 4060. A first straight lateral side 4062 and a second substantially straight lateral side 4064 can extend substantially perpendicular from the posterior side 4060 to a substantially straight anterior side 4066. In a particular embodiment, the anterior side 4066 and the posterior side 4060 are substantially the same length. Further, in a particular embodiment, the lateral sides 4062, 4064 are substantially the same length.

In a particular embodiment, the inferior component 4100 can include an inferior support plate 4102 that has an inferior articular surface 4104 and an inferior bearing surface 4106. In a particular embodiment, the inferior articular surface 4104 can be generally curved and the inferior bearing surface 4106 can be substantially flat. In an alternative embodiment, the inferior articular surface 4104 can be substantially flat and at least a portion of the inferior bearing surface 4106 can be generally curved.

As illustrated in FIG. 39 through FIG. 41, a depression 4108 extends into the inferior articular surface 4104 of the inferior support plate 4102. In a particular embodiment, the depression 4108 is sized and shaped to receive the projection 4008 of the superior component 4000. For example, the depression 4108 can have a hemi-spherical shape. Alternatively, the depression 4108 can have an elliptical shape, a cylindrical shape, or other arcuate shape.

Referring to FIG. 41, the depression 4108 can include a base 4120 and an inferior wear resistant layer 4122 affixed to, deposited on, or otherwise disposed on, the base 4120. In a particular embodiment, the base 4120 can act as a substrate and the inferior wear resistant layer 4122 can be deposited on the base 4120. Further, the base 4120 can engage a cavity 4124 that can be formed in the inferior support plate 4102. In a particular embodiment, the cavity 4124 can be sized and shaped to receive the base 4120 of the depression 4108. Further, the base 4120 of the depression 4108 can be press fit into the cavity 4124.

In a particular embodiment, the base 4120 of the depression 4108 can be made from graphite. Further, in a particular embodiment, the inferior wear resistant layer 4122 can be pyrolytic carbon that is deposited on the base 4120. As such, the base 4120 can be made from a material that can allow pyrolytic carbon to be deposited thereon. Thereafter, the base 4120 can be fitted into an inferior support plate 4102 made from one or more of the materials described herein. Accordingly, the inferior support plate 4102 may be made from a material that does not facilitate the deposition of pyrolytic carbon thereon.

Also, in a particular embodiment, the base 4120 can be roughened prior to the deposition of the pyrolytic carbon thereon. For example, the base 4120 can be roughened using a roughening process. In a particular embodiment, the roughening process can include acid etching; knurling; application of a bead coating, e.g., cobalt chrome beads; application of a roughening spray, e.g., titanium plasma spray (TPS); laser blasting; or any other similar process or method. Alternatively, the surface of the base 4120 on which the pyrolytic carbon is deposited can be serrated and can include one or more teeth, spikes, or other protrusions extending therefrom. The serrations of the base 4120 can facilitate anchoring of the pyrolytic carbon on the base 4120 and can substantially reduce the likelihood of delamination of the inferior wear resistant layer 4122 from the base 4120.

In a particular embodiment, the inferior wear resistant layer 4122 can have a thickness in a range of fifty micrometers to five millimeters (50 μm-5 mm). Further, the inferior wear resistant layer 4122 can have a thickness in a range of two hundred micrometers to two millimeters (200 μm-2 mm). In a particular embodiment, the serrations that can be formed on the surface of the base 4120 can have a height that is at most half of the thickness of the inferior wear resistant layer 4122. Accordingly, the likelihood that the serrations will protrude through the inferior wear resistant layer 4122 is substantially minimized.

Additionally, in a particular embodiment, a Young's modulus of the inferior wear resistant layer 4122 can be substantially greater than a Young's modulus of the base 4120. Also, a hardness of the inferior wear resistant layer 4122 can be substantially greater than a hardness of the base 4120. Further, a toughness of the inferior wear resistant layer 4122 can be substantially greater than a toughness of the base 4120. In a particular embodiment, the inferior wear resistant layer 4122 can be annealed immediately after deposition in order to minimize cracking of the inferior wear resistant layer. Also, the inferior wear resistant layer 4122 can be polished in order to minimize surface irregularities of the inferior wear resistant layer 4122 and increase a smoothness of the inferior wear resistant layer 4122.

FIG. 39 through FIG. 41 also show that the inferior component 4100 can include an inferior bracket 4148 that can extend substantially perpendicular from the inferior support plate 4502. Further, the inferior bracket 4148 can include a hole 4150. In a particular embodiment, a fastener, e.g., a screw, can be inserted through the hole 4150 in the inferior bracket 4548 in order to attach, or otherwise affix, the inferior component 4500 to an inferior vertebra.

The inferior bearing surface 4106 can be coated with a bone-growth promoting substance, e.g., a hydroxyapatite coating formed of calcium phosphate. Additionally, the inferior bearing surface 4106 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

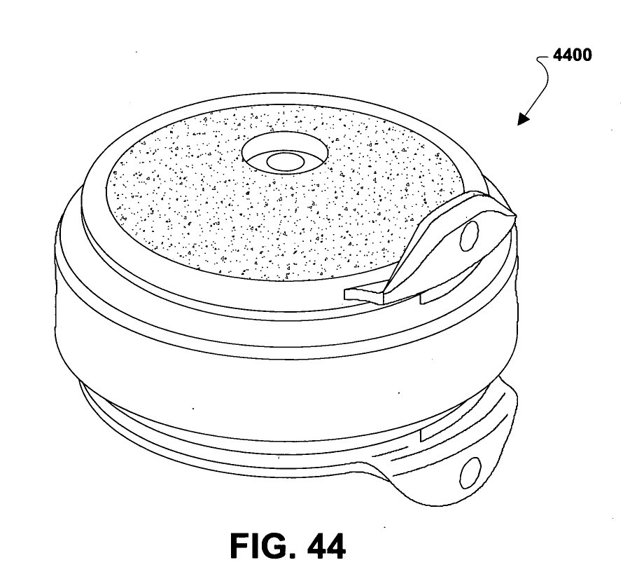

As illustrated in FIG. 43, the inferior component 4100 can be generally rectangular in shape. For example, the inferior component 4100 can have a substantially straight posterior side 4160. A first straight lateral side 4162 and a second substantially straight lateral side 4164 can extend substantially perpendicular from the posterior side 4160 to a substantially straight anterior side 4166. In a particular embodiment, the anterior side 4166 and the posterior side 4160 are substantially the same length. Further, in a particular embodiment, the lateral sides 4162, 4164 are substantially the same length.

In a particular embodiment, the overall height of the intervertebral prosthetic device 3900 can be in a range from fourteen millimeters to forty-six millimeters (14-46 mm). Further, the installed height of the intervertebral prosthetic device 3900 can be in a range from eight millimeters to sixteen millimeters (8-16 mm). In a particular embodiment, the installed height can be substantially equivalent to the distance between an inferior vertebra and a superior vertebra when the intervertebral prosthetic device 3900 is installed there between.

In a particular embodiment, the length of the intervertebral prosthetic device 3900, e.g., along a longitudinal axis, can be in a range from thirty millimeters to forty millimeters (30-40 mm). Additionally, the width of the intervertebral prosthetic device 3900, e.g., along a lateral axis, can be in a range from twenty-five millimeters to forty millimeters (25-40 mm). Moreover, in a particular embodiment, each bracket 4048, 4148 can have a height in a range from three millimeters to fifteen millimeters (3-15 mm).

### DESCRIPTION OF A SEVENTH EMBODIMENT OF AN INTERVERTEBRAL PROSTHETIC DISC

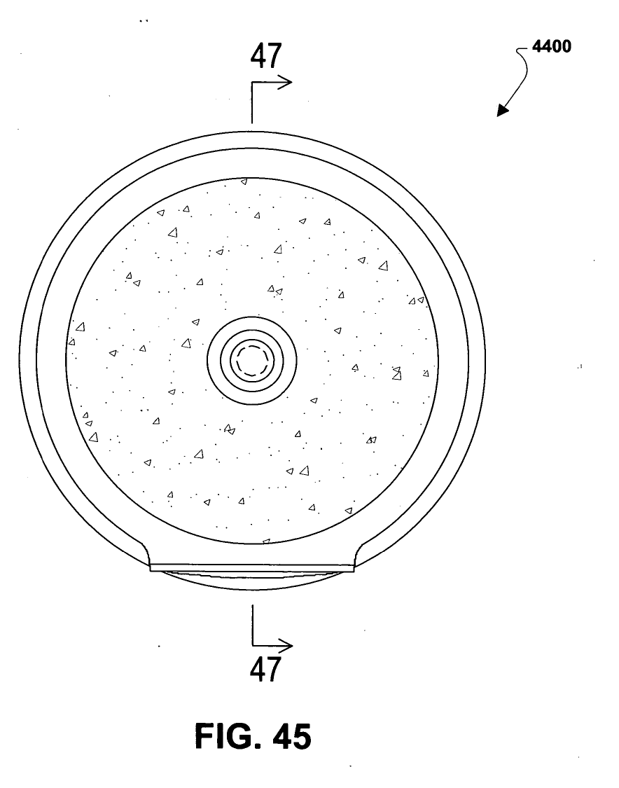

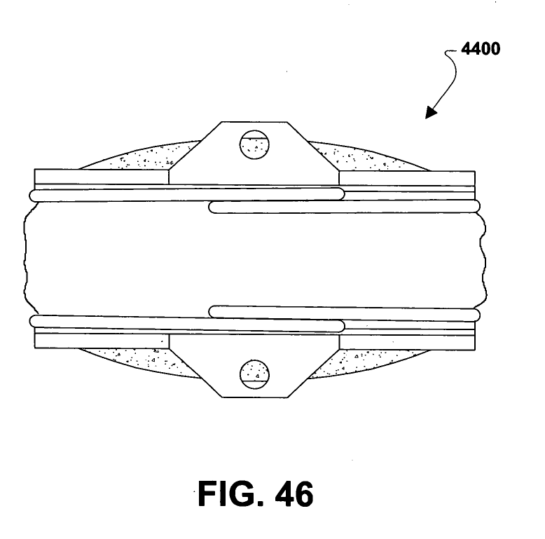

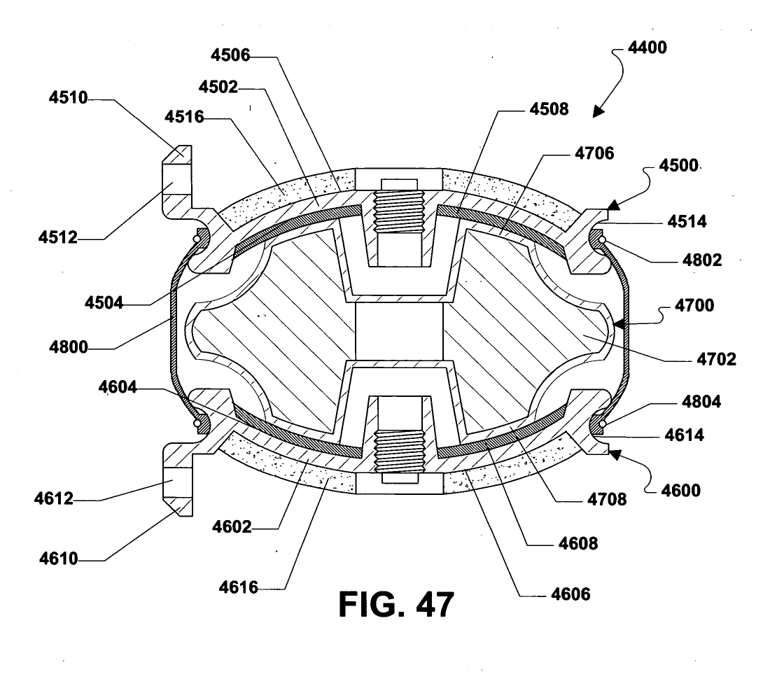

Referring to FIGS. 44 through 47, a seventh embodiment of an intervertebral prosthetic disc is shown and is generally designated 4400. As illustrated in FIG. 47, the intervertebral prosthetic disc 4400 can include a superior component 4500, an inferior component 4600, and a nucleus 4700 disposed, or otherwise installed, there between. In a particular embodiment, a sheath 4800 surrounds the nucleus 4700 and is affixed or otherwise coupled to the superior component 4500 and the inferior component 4600. In a particular embodiment, the components 4500, 4600 and the nucleus 4700 can be made from one or more biocompatible materials. For example, the materials can be metal containing materials, polymer materials, or composite materials that include metals, polymers, or combinations of metals and polymers. Additionally, the biocompatible materials can include, or contain, an inorganic carbon-based material, such as graphite.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 4500, 4600 can be made from any other substantially rigid biocompatible materials.

In a particular embodiment, the superior component 4500 can include a superior support plate 4502 that has a superior articular surface 4504 and a superior bearing surface 4506. In a particular embodiment, the superior support plate 4502 can be generally rounded, generally cup shaped, or generally bowl shaped. Further, in a particular embodiment, the superior articular surface 4504 can be generally rounded or generally curved and the superior bearing surface 4506 can be generally rounded or generally curved.

As illustrated in FIG. 47, a superior wear resistant layer 4508 is disposed on, or otherwise affixed to, the superior bearing surface 4506. In a particular embodiment, the superior wear resistant layer 4508 can be shaped to match the shape of the superior support plate 4502. Additionally, in a particular embodiment, the superior wear resistant layer 4508 is made from a substantially wear resistant material. In a particular embodiment, the superior wear resistant layer 4508 can be pyrolytic carbon.

FIG. 47 also shows that the superior support plate 4502 can include a superior bracket 4510 that can extend substantially perpendicular from the superior support plate 4502. The superior bracket 4510 can include a hole 4512. In a particular embodiment, a fastener, e.g., a screw, can be inserted through the hole 4512 in the superior bracket 4510 in order to attach, or otherwise affix, the superior component 4500 to a superior vertebra.

Moreover, the superior support plate 4502 includes a superior channel 4514 established around the perimeter of the superior support plate 4502. In a particular embodiment, a portion of the sheath 4800 can be held within the superior channel 4514 using a superior retaining ring 4802.

As depicted in FIG. 47, the superior support plate 4502 can include a bone growth promoting layer 4516 disposed, or otherwise deposited, on the superior bearing surface 4506. In a particular embodiment, the bone growth promoting layer 4516 can include a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

In a particular embodiment, the inferior component 4600 can include an inferior support plate 4602 that has an inferior articular surface 4604 and an inferior bearing surface 4606. In a particular embodiment, the inferior support plate 4602 can be generally rounded, generally cup shaped, or generally bowl shaped. Further, in a particular embodiment, the inferior articular surface 4604 can be generally rounded or generally curved and the inferior bearing surface 4606 can be generally rounded or generally curved.

As illustrated in FIG. 47, an inferior wear resistant layer 4608 is disposed on, or otherwise affixed to, the inferior bearing surface 4606. In a particular embodiment, the inferior wear resistant layer 4608 can be shaped to match the shape of the inferior support plate 4602. Additionally, in a particular embodiment, the inferior wear resistant layer 4608 is made from a substantially wear resistant material. In a particular embodiment, the inferior wear resistant layer 4608 can be pyrolytic carbon.

FIG. 47 also shows that the inferior support plate 4602 can include an inferior bracket 4610 that can extend substantially perpendicular from the inferior support plate 4602. The inferior bracket 4610 can include a hole 4612. In a particular embodiment, a fastener, e.g., a screw, can be inserted through the hole 4612 in the inferior bracket 4610 in order to attach, or otherwise affix, the inferior component 4600 to an inferior vertebra.

Moreover, the inferior support plate 4602 includes an inferior channel 4614 established around the perimeter of the inferior support plate 4602. In a particular embodiment, a portion of the sheath 4800 can be held within the inferior channel 4614 using an inferior retaining ring 4804.

As depicted in FIG. 47, the inferior support plate 4602 can include a bone growth promoting layer 4616 disposed, or otherwise deposited, on the inferior bearing surface 4606. In a particular embodiment, the bone growth promoting layer 4616 can include a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

As depicted in FIG. 47, the nucleus 4700 can be generally toroid shaped. Further, the nucleus 4700 includes a core 4702 and an outer wear resistant layer 4704. In a particular embodiment, the core 4702 of the nucleus can be made from one or more biocompatible materials. For example, the materials can be metal containing materials, polymer materials, or composite materials that include metals, polymers, or combinations of metals and polymers. Additionally, the biocompatible materials can include, or contain, an inorganic carbon-based material, such as graphite.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

In a particular embodiment, at least a portion of the outer wear resistant layer 4704 of the nucleus can be made from a substantially wear resistant material. Further, the substantially wear resistant material can be pyrolytic carbon.

As illustrated in FIG. 47, the outer wear resistant layer 4704 of the nucleus 4700 can include a superior portion 4706 and an inferior portion 4708. In a particular embodiment, the superior portion 4706 of the outer wear resistant layer 4704 of the nucleus 4700 can be curved to match the curvature of the superior wear resistant layer 4508 that is disposed on, or otherwise affixed to, the superior bearing surface 4506. Further, the superior portion 4706 of the outer wear resistant layer 4704 of the nucleus 4700 can slide relative to the superior wear resistant layer 4508 and can allow relative motion between the superior component 4500 and the nucleus 4700.

Also, in a particular embodiment, the inferior portion 4708 of the outer wear resistant layer 4704 of the nucleus 4700 can be curved to match the curvature of the inferior wear resistant layer 4608 that is disposed on, or otherwise affixed to, the inferior bearing surface 4606. Further, the inferior portion 4708 of the outer wear resistant layer 4704 of the nucleus 4700 can slide relative to the inferior wear resistant layer 4608 and can allow relative motion between the inferior component 4600 and the nucleus 4700.

In a particular embodiment, the entire outer wear resistant layer 4704 of the nucleus 4700 can be made from the substantially wear resistant material. Alternatively, the superior portion 4706 of the outer wear resistant layer 4704, the inferior portion 4708 of the outer wear resistant layer 4704, or a combination thereof can be made from the substantially wear resistant material.

### CONCLUSION

With the configuration of structure described above, the intervertebral prosthetic disc according to one or more of the embodiments provides a device that may be implanted to replace a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion there between.

In a particular embodiment, the wear resistant layers provided by one or more of the intervertebral prosthetic discs described herein can limit the wear of the moving components caused by motion and friction. Further, the wear resistant layers provided by one or more of the intervertebral prosthetic discs described herein can increase the life of an intervertebral prosthetic disc. Accordingly, the time before the intervertebral prosthetic disc may need to be replaced can be substantially increased. Further, the wear resistant layers described herein can reduce the occurrence and amount of wear debris, which could otherwise produce undesired or deleterious effects on collateral systems.

Additionally, in a particular embodiment, a Young's modulus of the wear resistant layers can be substantially greater than a Young's modulus of a underlying material on which the wear resistant layers can be disposed. Also, a hardness of the wear resistant layers can be substantially greater than a hardness of the underlying material on which the wear resistant layers can be disposed. Further, a toughness of the wear resistant layers can be substantially greater than a toughness of an underlying material on which the wear resistant layers can be disposed.

The above-disclosed subject matter is to be considered illustrative, and not restrictive, and the appended claims are intended to cover all such modifications, enhancements, and other embodiments that fall within the true spirit and scope of the present invention. For example, it is noted that the components in the exemplary embodiments described herein are referred to as “superior” and “inferior” for illustrative purposes only and that one or more of the features described as part of or attached to a respective half may be provided as part of or attached to the other half in addition or in the alternative. Thus, to the maximum extent allowed by law, the scope of the present invention is to be determined by the broadest permissible interpretation of the following claims and their equivalents, and shall not be restricted or limited by the foregoing detailed description.

1. An intervertebral prosthetic disc configured to be installed within an intervertebral space between a superior vertebra and an inferior vertebra, the intervertebral prosthetic disc comprising:
an inferior component having a depression formed therein; and
a superior component having a projection extending therefrom, wherein the projection is configured to movably engage the depression and allow relative motion between the inferior component and the superior component and wherein the projection includes a superior wear resistant layer configured to engage the depression.

2. The intervertebral prosthetic disc of claim 1, wherein the projection includes a base and wherein the superior wear resistant layer is deposited on the base.

3. The intervertebral prosthetic disc of claim 2, wherein the superior component includes a cavity sized and shaped to receive the base of the projection.

4. The intervertebral prosthetic disc of claim 3, wherein the base of the projection is installed within the cavity formed in the superior component.

5. The intervertebral prosthetic disc of claim 2, wherein a Young's modulus of the superior wear resistant layer is greater than a Young's modulus of the base.

6. The intervertebral prosthetic disc of claim 2, wherein a hardness of the superior wear resistant layer is greater than a hardness of the base.

7. The intervertebral prosthetic disc of claim 2, wherein a toughness of the superior wear resistant layer is greater than a toughness of the base.

8. The intervertebral prosthetic disc of claim 1, wherein the inferior component further comprises an inferior wear resistant layer deposited within the depression wherein the inferior wear resistant layer is configured to engage the superior wear resistant layer.

9. The intervertebral prosthetic disc of claim 8, wherein the depression includes a base and wherein the inferior wear resistant layer is deposited within the base.

10. The intervertebral prosthetic disc of claim 9, wherein the inferior component includes a cavity size and shaped to receive the base of the depression.

11. The intervertebral prosthetic disc of claim 10, wherein the base of the depression is installed within the cavity formed in the inferior component.

12. The intervertebral prosthetic disc of claim 11, wherein the superior component further comprises a superior bracket extending therefrom, wherein the superior bracket is configured to be attached to the superior vertebra.

13. The intervertebral prosthetic disc of claim 1, wherein the inferior component further comprises an inferior bracket extending therefrom, wherein the inferior bracket is configured to be attached to the inferior vertebra.

14. The intervertebral prosthetic disc of claim 13, wherein the base of the depression comprises graphite.

15. The intervertebral prosthetic disc of claim 1, wherein the superior wear resistant layer comprises pyrolytic carbon.

16. The intervertebral prosthetic disc of claim 15, wherein the superior component, the inferior component, or a combination thereof comprises a biocompatible material.

17. The intervertebral prosthetic disc of claim 16, wherein the biocompatible material is a pure metal, a metal alloy, a polymer, a ceramic, a carbon-based material, or a combination thereof.

18. The intervertebral prosthetic disc of claim 17, wherein the pure metal comprises titanium.

19. The intervertebral prosthetic disc of claim 17, wherein the metal alloy comprises stainless steel, cobalt-chrome-molybdenum alloy, titanium alloy, or a combination thereof.

20. The intervertebral prosthetic disc of claim 17, wherein the polymer comprises polyurethane, polyolefin, polyaryletherketone (PAEK), silicone, hydrogel, or a combination thereof.

21. The intervertebral prosthetic disc of claim 20, wherein the polyolefin comprises polypropylene, polyethylene, halogenated polyolefin, flouropolyolefin, or a combination thereof.

22. The intervertebral prosthetic disc of claim 20, wherein the polyaryletherketone (PAEK) comprises polyetherketone (PEK), polyetheretherketone (PEEK), polyetherketoneketone (PEKK), polyetherketoneetherketoneketone (PEKEKK), or a combination thereof.

23. The intervertebral prosthetic disc of claim 17, wherein the carbon-based material comprises graphite.

24. An intervertebral prosthetic disc configured to be installed within an intervertebral space between a superior vertebra and an inferior vertebra, the intervertebral prosthetic disc comprising:
an inferior component having a depression formed therein; and
a superior component having a projection extending therefrom, wherein the projection comprises a base and a wear resistant layer disposed on the base, wherein the wear resistant layer is configured to movably engage the depression and allow relative motion between the inferior component and the superior component.

25-41. (canceled)

42. An intervertebral prosthetic disc configured to be installed within an intervertebral space between a superior vertebra and an inferior vertebra, the intervertebral prosthetic disc comprising:
an inferior component having an inferior depression formed therein;
a superior component having a superior depression formed therein; and
a nucleus disposed between the inferior component and the superior component, wherein the nucleus includes a superior wear resistant layer and an inferior wear resistant layer, wherein the superior wear resistant layer of the nucleus is configured to movably engage the superior depression and wherein the inferior wear resistant layer of the nucleus is configured to movably engage the inferior depression.

43-46. (canceled)

47. An intervertebral prosthetic disc configured to be installed within an intervertebral space between a superior vertebra and an inferior vertebra, the intervertebral prosthetic disc comprising:
an inferior component having an inferior projection extending therefrom;
a superior component having a superior projection extending therefrom; and
a nucleus disposed between the inferior component and the superior component, wherein the nucleus includes a superior depression having a superior wear resistant layer therein and an inferior depression having an inferior wear resistant layer therein, wherein the superior wear resistant layer of the nucleus is configured to movably engage the superior projection and wherein the inferior wear resistant layer of the nucleus is configured to movably engage the inferior projection.

48-50. (canceled)

51. An intervertebral prosthetic disc configured to be installed within an intervertebral space between a superior vertebra and an inferior vertebra, the intervertebral prosthetic disc comprising:
an inferior component;
a superior component; and
a generally toroidal nucleus disposed between the inferior component and the superior component, wherein the nucleus includes a core and an outer wear resistant layer disposed on the core, wherein the outer wear resistant layer of the core is configured to movably engage the inferior component and the superior component.

52-57. (canceled)
